(19) United States
(12) Patent Application Publication
(10) Pub. No.: US 2008/0221013 A1
(43) Pub. Date: Sep. 11, 2008

(54) Neurobiological compositions

(76) Inventors: Julie Miwa, New York, NY (US); Nathaniel Heintz, Pehlem Manor, NY (US); Ayse Begum Tekinay, Clifton, NJ (US)

Correspondence Address: Julie Michiko Miwa, 504 E. 63rd St. #34S, New York, NY, 10065, US

(21) Appl. No.: 11/899,406

(22) Filed: Sep. 4, 2007

Related U.S. Application Data
(60) Provisional application No. 60/841,697, filed on Sep. 2, 2006.

(51) Int. Cl. A61K 38/00; A61P 25/00

(52) U.S. Cl. 514/2

(57) ABSTRACT

Disclosed herein are compositions and methods for the treatment or prevention of neurological disorders using lynx compounds. The invention further discloses compositions and methods for the modulation of acetylcholine receptor activity.

### RELATED APPLICATIONS

This application claims the benefit of the U.S. Provisional Application 60/841,697, filed Sep. 2, 2006, entitled “lynx family of modulators compositions and methods of use thereof.”

### STATEMENT REGARDING FEDERALLY SPONSORED RESEARCH OF DEVELOPMENT

N/A

### THE NAMES OF THE PARTIES TO A JOINT RESEARCH AGREEMENT

N/A

### REFERENCE TO A SEQUENCE LISTING CD

1 compact disc to be incorporated-by-reference:

file name: application 11-988,406-ST25.txt

created: Jan. 24, 2008

size: 46 kb

### BACKGROUND OF THE INVENTION

1. Field of the Invention

The field of this invention contemplates the treatment of cognitive disorder using the composition of the invention wherein the composition of the invention is a polypeptide, DNA or RNA molecule from the lynx1a, lynx1b, lynxc, lynx2 or lynx3 sequences. The cognitive disorders emcompass cognitive disorders, demetias, anxiety disorder, mood disorder, pain, neuropathis pain, epilepsies such as ADNFLE and addiction. The field of this invention relates generally to compositions and methods for the treatment or prevention of neurological and other disorders as well as for the modulation of acetylcholine receptor activity.

2. Description of the Related Art

Nicotinic acetylcholine receptors have been shown to contribute to such diverse physiological functions as learning and memory, addiction, antinociception, attention and mood disorders such as anxiety, and depression. (Reviewed in Rezvani and Levin, 2001, Biol Psych. 49, 258-67, Picciotto et al., 2000, Neuropsychopharmacol. 22, 451-465). nAChRs are gated by the neurotransmitter (NT) acetylcholine (ACh), which is released from axonal terminals distributed throughout the brain, as well as by the drug nicotine, which is the primary addictive agent in tobacco (US Department of Health and Human Services, 1988,U.S. Government Printing Office). The importance of maintaining the proper balance of cholinergic signaling has been well documented. Reductions in nAChR levels or activity have been correlated with Alzheimer's disease, schizophrenia, Parkinson's disease, neurodegeneration and dementia (Lindstrom, 1997, Mol. Neurobiol. 15, 193-222), while in other cases over-activation of nAChRs can be detrimental. High doses of nicotine can induce epilepsy (Damaj et al., 1999, J. Pharmacol. Exp. Ther. 291, 1284-1291) and cause cell death (Abrous et al., 2002, J. Neurosci. 22, 3656-3662), and knockin mice with hyperactive alpha7 or alpha4 nAChRs exhibit lower seizure thresholds (Fonck et al., 2003, J. Neurosci. 23, 2582-2590, Fonck et al., 2005, J. Neurosci. 25, 11396-11411, Broide et al., 2002, Mol. Pharmacol. 61, 695-705) and suffer from neuronal loss (Orr-Urtreger et al., 2000, J. Neurochem. 74, 2154-2166). Even subtle alterations in nAChR activity, such as those resulting from changes in desensitization kinetics, can have important consequences on cholinergic function (Dani et al., 2000, Eur. J. Pharmacol. 393, 31-38, Wooltorton et al., 2003, J. Neurosci. 23, 3176-3185). One form of epilepsy, ADNFLE, caused by mutations in the widely-expressed nAChR subunits, alpha4 or beta2, results from altered desensitization and gating properties of nAChRs (Kuryatov, et al., 1997, J. Neurosci. 17, 9035-9047). Therefore, maintenance of cholinergic tone is critical to normal CNS function.

Nicotinic acetylcholine receptors are a large family of related genes all gated by the same neurotransmitter, acetylcholine, and able to bind to and be gated by the exogenous compound nicotine. Neuronal nicotinic acetylcholine receptors (nAChRs) are pentameric cationic channels gated by a ligand: acetylcholine (ACh), the endogenous ligand, and exogenous molecules such as nicotine, the most widespread drug of abuse. nAChRs form a heterogeneous family of pentameric oligomers expressed in various brain areas and in ganglia, whose subtype diversity is a consequence of the combinations of subunits encoded by at least 12 different genes divided into two subfamilies: nine alpha or ligand binding subunits (alpha2-alpha10) and three beta or structural subunits (beta2-beta4). Both alpha and beta subunits can contribute to the receptor pharmacological properties though homoligomeric receptors can exist, and regardless of composition, each receptor has five ACh-binding sites per receptor molecule, one on each subunit. Because of the diversity of receptor species, subunit compositions at any given site in vivo is not well understood due. Therefore, while the effects of nicotine and/or nicotinic receptor activation have been linked with a wide array of physiological disorders, the contribution of an individual subunit to a given disorder is in most cases poorly understood.

CNS therapeutic applications for the acetylcholine receptors include anticholinergic agents in the treatment of schizophrenia, Alzheimer's, and Parkinson's disease. Because cholinergic dysfunction is associated with the cognitive impairment in Alzheimer's disease nicotinic acetylcholine receptors have been implicated as a potential therapeutic target for Alzheimers as well as in other memory learning and cognitive disorders, including Lewy Body dementia attention deficit disorder.

It would be desirable to provide a method for the prevention and treatment of a condition or disorder by administering a cholinergic activator to a patient susceptible to or suffering from such a condition or disorder, or to delay or prevent the onset, shorten the duration of, or ameliorate or reverse the symptoms of those disorders by the administration of a pharmaceutical agent active on nicotinic receptors, that has a beneficial effect (e.g., upon the functioning of the CNS), but that does has the least possible associated side effects. Therefore specific activation of select subspecies of nicotinic receptors would be highly beneficial, in that said compound has the effect of activating nicotinic receptor function when employed in an amount sufficient to affect the functioning of the CNS, but does not significantly affect those receptor subtypes which have the potential to induce undesirable side effects.

The ly-6/uPAR family member lynx1 (Miwa et al., 1999, Neuron 23, 105-114), can form stable associations with nAChRs and alter their function in vitro (Ibanez-Tallon et al., 2002, Neuron 33, 893-903). lynx1, an evolutionary precursor to snake venom toxins, shares structural characteristics with toxins such as alpha- and kappa-bungarotoxins, which bind tightly to nAChRs and inhibit their activation. When co-expressed with lynx1, alpha4beta2nAChRs are less sensitive to ACh, have a higher EC50, display more rapid desensitization, and recover more slowly from desensitization. Single channel studies of alpha4beta2 receptors indicate that lynx1 shifts the distribution of channel openings toward a faster inactivating species with more uniform, larger amplitude currents. lynx1 modulates acetylcholine receptor function in the presence of its natural ligand, which demonstrates that lynx1 acts as an allosteric modulator of nicotinic acetylcholine receptors. lynx1 co-localizes and co-immunoprecipitates with alpha7 and beta2 nAChR subunits, are strong indicators that lynx1 has a critical role in modulating cholinergic activity in vivo. Because the snake venom toxins have diverse, but specific actions, on distinct receptor subtypes, the nature of the specific interaction of the lynx1 polypeptide and its functional effect in vivo isn't likely to be deduced (Ibanez-Tallon et al, 2004, Neuron 43, 305-311). A related gene, lynx2, has distinct and overlapping expression patterns with lynx1 in the brain (Dessaud et al, 2006, Mol. Cell. Neurosci. 2006, 31, 232-242), and is also functionally related (see Example 1 below).

The Ly-6/uPAR superfamily of proteins containing a characteristic eight or ten cysteine motif, in which the carboxy terminus of the mature protein contains CCXXXXCN as part of this motif, (Gumley et al., 1995, Immunogenetics 42, 221-224, Fleming et al., 1993, J. Immunol. 150, 5379-5390, Ploug and Ellis, 1994, FEBS Lett. 349, 163-168). Members of the ly-6/uPAR superfamily of genes include Retinoic acid-induced gene E (RIG-E), the E48 antigen, Ly-6H, the PSCA, TSA-1, CD59, lynx1, lynx2 and uPAR, SLURP-1 (Adermann et al, 1999, Protein Sci. 8, 810-819 and SLURP-2 (Arredondo, et al., 2006, J. Cell Physiol. 208, 238-45, Kawashima et al., 2007, Life Sci., 80, 2314-2319)). Disulfide bonding occurs between these conserved cysteine residues which is results in the characteristic structural motif termed the toxin fold. (Rees et al., 1987, Proc. Natl. Acad. Sci. 84, 3132-3136). There are two main families of proteins within this diverse family, the ly-6 proteins, and snake venom toxins. The structural similarity of these diverse groups of proteins were confirmed through crystallographic analysis of one member of each group, aBTX and CD59 (Love and Stroud, 1986, Pro. Engineer. 1, 37-46, Fletcher et al, 1994, Structure, 2, 185-199).

Nicotinic acetylcholine receptors (nAChRs) affect a wide array of biological processes including learning and memory, attention, and addiction. lynx1 modulates nAChR function in vitro by altering agonist sensitivity and desensitization kinetics. Generation of lynx1 null mutant mice, indicates that lynx1 modulates nAChR signaling in vivo. Its loss decreases the EC₅₀ for nicotine by 10-fold, decreases receptor desensitization, elevates intracellular calcium levels in response to nicotine, and enhances synaptic efficacy. lynx1 null mutant mice exhibit enhanced performance in specific tests of learning and memory. Consistent with reports that mutations resulting in hyper-activation of nAChRs can lead to neurodegeneration, aging lynx1 null mutant mice exhibit a vacuolating degeneration that is exacerbated by nicotine and ameliorated by null-mutations in nAChRs. Therefore, lynx1 functions as an allosteric modulator of nAChR activity in vivo, balancing neuronal activity and survival in the CNS (Miwa et al., 2006, Neuron, 51, 587-600, or see Example 2).

Disclosed herein is the involvement of the lynx1 gene in cognitive disorders and neurodegenerative disorders. Through the generation of a null mutation of the lynx1 gene through homologous recombination in ES cells (See example 2), we determine that the lynx1 gene is involved in synaptic signaling, learning and memory, calcium homeostasis. Removal of the lynx1 gene leads to cognitive effects, synaptic effects, and nicotine hypersensitivity, and causes neurons to be more susceptible to damage and glutamate mediated toxicity.

One aspect of the invention is the use of lynx3, or derivative thereof, as a therapeutic tool in the treatment of said physiological disorder. lynx3 exhibits modulatory capacity on nAChRs (see Example 1).

### BRIEF SUMMARY OF THE INVENTION

The invention described herein contemplates the treatment of neurological disorders in an individual by administration of an effective dose of lynx1, whereby lynx1 consists of a sequence as set forth in SEQ ID NO:1, or can be fragment, or related polypeptide of lynx1, and wherein a composition of lynx1 acts as a biological therapeutic for the treatment of said neurological disorder.

In one embodiment of the present invention, based on the selective nature of lynx1 action on subtypes of nicotinic acetylcholine receptors, and based the specific nature of altering activity of these subtypes, the lynx1 polypeptide, RNA, or DNA is administered to an individual in order to affect physiological processes in vivo.

This invention also describes methods for delaying the onset of, or preventing the start of, said neurological disorder in a subject by administration of an effective dose of lynx1 or a related gene, transcript, or polypeptide whereby lynx1 is administered to an individual suffering from said disorder, and whereby lynx1 is administered as a biological therapeutic agent.

Within the description of this invention, also includes methods of providing protection to a subject by administration an effective dose of lynx1 or a related gene, transcript, or polypeptides to a subject suffering from a neurological disorder, where such protection prevents a neurological disorder caused by dysfunction of an acetylcholine receptor.

Another aspect of the present invention is the administration of lynx1 for the treatment of neurological disorders in which the etiology of the disorder is not the dysfunction of a nicotinic acetylcholine receptor, per se, but wherein alterations in the level of activity of a nicotinic acetylcholine receptor can lessen the severity, shorten the duration, or ameliorate the symptoms of the disorder.

Compositions described herein also comprise an effective dose of lynx1 DNA, RNA, or polypeptide, or combination thereof, with or without a carrier or an excipient, preferably but not exclusively formulated as described below, where the composition administered to a subject has the capability to functionally modulate the activity of an alpha7 nicotinic acetylcholine receptor of an alpha7 nicotinic acylcholine receptor-subunit containing receptor, or the function of a related protein.

Compositions described herein also comprise an effective dose of lynx1 DNA, RNA, or polypeptide, or a combination thereof, with or without a carrier or an excipient, preferably but not exclusively formulated as described below, where the composition administered to a subject has the capability to functionally modulate the activity of a beta2 nicotinic acetylcholine receptor subunit-containing receptor channel, or of a related polypeptide.

In preferred embodiments of the invention, treatments may be used for neurological disorders which may or may not be caused by dysfunction of an acetylcholine receptor. Neurological disorders include but are not limited to neurodegenerative disorders such as Alzheimer's disease, Parkinson's disease and cognitive impairments; neuropsychiatric disorders such as schizophrenia, and disorders such as addiction, pain, and neuropathic pain

The lynx1 polypeptide of the invention, or fragment of lynx1 polypeptide can be administered to the subject in a mature form. The mature form of lynx1 as described herein includes amino acids 21-129 of lynx1, isoform a (SEQ ID NO:1), amino acids 23-95 for lynx1, isoform b (ADD AND REFERENCE SEQUENCE), and amino acids 23-92 for lynx1, isoform c (SEQ ID NO:2). This corresponds to amino acids 21-92 in the mouse lynx1 gene (SEQ ID NO:3).

In another embodiment of the invention, based on the specific nature of individual lynx DNAs, RNAs, or polypeptides with respect to expression profile, the specificity of receptor binding, and in vivo function, specific therapeutics for the treatment of neurological and other disorders or conditions are produced and administered to an individual. lynx1 derivatives may be used as treatments, as non-limiting examples, for dementia, pain and epilepsy functions, whereas lynx2 (SEQ ID NO:4) may be used, as a non-limiting example, as a treatment for anxiety. Therefore the invention allows for multiple neurological therapies, utilizing the different pharmacological profiles of each of the lynx family members.

In yet another embodiment of the invention, based on lynx3 (SEQ ID NO:18 and SEQ ID NO:6) exhibits modulatory capacity on nAChRs is the use of lynx3, SEQ ID NO:6, or derivative thereof, as a therapeutic tool in the treatment of neurophysiological disorders.

### BRIEF DESCRIPTION OF THE DRAWINGS

FIG. 1. A and B: in situ hybridization experiments demonstrate a complementary expression patterns of the lynx1 and lynx2 genes. C-G: alpha4beta2 nAChR proteins form stable associations with lynx1, lynx2 lynx3 gene products, as demonstrated in co-immunoprecipitation experiments in transiently transfected cells.

FIG. 2. lynx polypeptides are nAChR modulatory polypeptides, as assessed by voltage-clamp recordings in a xenopus ooctye expression system. lynx1, lynx2 and lynx3 enhance desensitization of ACh-evoked currents mediated through a4b2 nAChRs in oocytes. A. Representative recordings of voltage clamped oocytes expressing a4b2 nAChRs alone, or in combination with lynx1, lynx2, lynx3 and ly6H. The inward currents were evoked by 20 sec periods of superfusion (horizontal calibration bar) with external saline containing 1 mM ACh. ACh evoked responses in oocytes coexpressing a4b2 nAChRs with lynx1, lynx2 or lynx3 showed significantly faster desensitization during agonist application immediately after the initial peak. ly6h had no effect on desensitization when coexpressed with a4b2 receptors. B and C. The differences in desensitization are shown with bar graphs. As described in (2), two exponentials equations were fitted to the desensitization currents during ACh application. Using these equations, fast (B) and slow (C) time constants were calculated and the average values of these constants for ACh responses are shown in B and C. In oocytes coexpressing a4b2 nAChRs with lynx1, lynx2 or lynx3, the fast time constant is significantly faster, while the slow time constant during the plateau phase remained the same. Both constants are unaffected in oocytes coexpressing Ly6H.

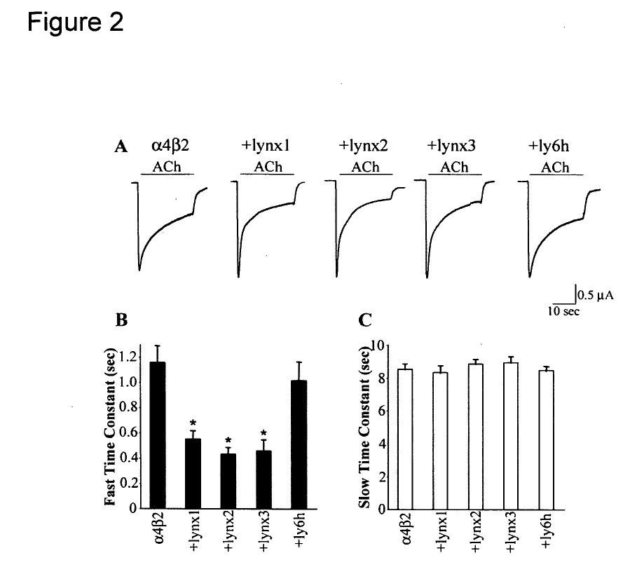

FIG. 3A. A schematic diagram of the DNA construct is shown used for the generation of a lynx1 null mutant mouse, with a loss of the lynx1 polypeptide.

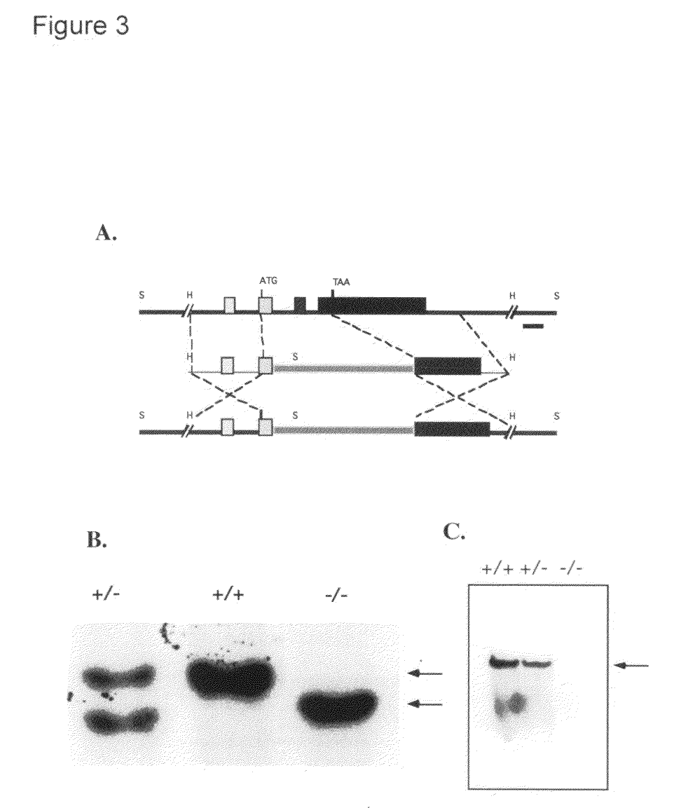

FIG. 4. Nicotine-induced currents in lynx1 null mutant mice demonstrate hypersensitivity to agonist in whole cell recordings in brain slices. These data indicate that nAChRs are altered in the absence of lynx 1, and that lynx1 is a critical component of the nAChR complex in the correct functioning of these receptors.

FIG. 5. lynx1 regulated calcium homeostasis in neurons, removal causes elevations in calcium and hypersensitivity to nicotine.

FIG. 6 shows altered synaptic responses in brain slice recordings in response to lynx1 removal, demonstrating that lynx1 is involved in regulated pre-synaptic release of neurotransmitter, and synaptic efficacy in the brain.

FIG. 7 shows enhancement of associative learning ability in lynx1 null mutant mice observed in fear conditioning assays. nAChR activation has been shown to be an important component of specific aspects of learning and memory. These data are suggestive of a specific effect of lynx1 on associative fear learning as compared to either unconditioned fear or contextual memory.

FIG. 8 shows enhancements in nicotine-mediated motor learning performance in lynx1 null mutant mice, and hypersensitivity to nicotine in vivo.

FIG. 9 shows nicotine-mediated neuroprotection against glutamate toxicity is abolished in primary cortical cultures from lynx1^{-/-} mice, demonstrating the importance of lynx in maintaining neuronal health.

FIG. 10. Degeneration in lynx1 null mutant mice within the dorsal striatum.

FIG. 11. Degeneration in lynx1 null mutant mice within the cerebellum.

FIG. 12. lynx1 null mutant mice is age-dependent, is accelerated with nicotine exposure, and rescued by null mutation in nAChR subunits.

FIG. 13. lynx1 null mutant mice display greater nicotine-mediated antinociception, and increased sensitivity to nicotine.

FIG. 14. lynx1 null mutant mice are more sensitive to nicotine induced seizures that wt mice.

### DETAILED DESCRIPTION OF THE INVENTION

### Summary

The invention described herein comprises methods for treating a physiological disorders in subjects. These could include neurological disorders including cognitive, mood, anxiety, addictive disorders and neurodegenerative disorders. The invention is practiced by use of the lynx1 composition, or derivatives thereof, for the treatment of these diseases and disorders. Members of the lynx family of proteins have been shown to alter the function of nicotinic acetylcholine receptors and therefore through this action can treat, ameliorate or improve the symptoms of a subject suffering from one of the above disorder. This invention also included the family members lynx2 and lynx3, in which polypeptides with the lynx2 and lynx3 compositions, or derivatives thereof, could be used to treat cognitive impairments such as those listed above.

### Compositions of the Invention

### Polypeptides of the Invention

Polypeptides of the instant invention include human lynx1a (SEQ ID NO:1), lynx1b, lynx1c (SEQ ID NO:2), mouse lynx1 (SEQ ID NO:3), human lynx2 (SEQ ID NO:4), mouse lynx2 (SEQ ID NO:5) human lynx3 (SEQ ID NO:6), and mouse lynx3 (SEQ ID NO:7) as described in detail below. DNA sequences as they encode for these polypeptides, and as they belong into the class of cysteine rich small molecules of the ly6 superfamily, a family of evolutionarily related genes, are also provided, including lynx1 variant 1 (SEQ ID NO:8), human lynx1 variant 2 (SEQ ID NO:9), human lynx1 variant 3 (SEQ ID NO:10), human lynx1 variant 4 (SEQ ID NO:11), lynx1 variant 5 (SEQ ID NO:19), mouse lynx1 (SEQ ID NO:12), human lynx2, variant 1 (SEQ ID NO:13), human lynx2, variant 2 (SEQ ID NO:14), human lynx2, variant 3 (SEQ ID NO:15), mouse lynx2 (SEQ ID NO:16), human lynx3 (SEQ ID NO:17), mouse lynx3 (SEQ ID NO:18), and may be collectively referred to as lynx DNA or lynx nucleotide sequences. By virtue of disulfide bonding between these cysteine residues, members of this family adopt a structure termed the three finger fold, or toxin fold. Polypeptides within the scope of the invention bind nicotinic acetylcholine receptors, variants, fragments, derivatives, conjugates, multimers, or fusions thereof, and thereby modulate receptor activity, downstream signaling, expression of genes responsive to polypeptides of the invention, and wider physiological effects mediated by binding of polypeptides of the invention to a receptors, and upon administration to an individual suffering from one of the disorders, conditions and/or diseases responsive to polypeptides of the invention thereby affect to the duration, severity and/or symptoms of the disorders, conditions and/or diseases described below.

Polypeptides that can be applied by the methods described herein consist of polypeptides where lynx polypeptides include any polypeptides or protein products of the listed classification of lynx1, lynx1a, lynx1b, lynx1c, lynx2 or lynx3 genes in any organisms, including prokaryotic, eukaryotic, mono-cellular, multi-cellular, animal, plant, fungus, vertebrate, invertebrate, mammalian, human, simian, monkey, murine, rat, mouse, porcine, bovine, feline, equine, canine, avian, insect, fruit fly, firefly, nematode, and any other biological species or beings. Polypeptides refer to any of the polypeptides or protein products of the genes comprising the sequences of SEQ ID 8 through SEQ ID 19, as well as derivatives thereof, where derivatives are defined as variants, fragments, conjugates, multimers, point mutants, or fusion proteins thereof. Fragments are defined as the polypeptide sequence of the “mature” lynx proteins, that is the lynx polypeptide sequence minus the cleaved signal sequence and the cleaved GPI-consensus sequence and its neighboring aspargine reside. For human lynx1a polypeptide, this lynx1 fragment refers to the polypeptide from amino acids 21-129 for human lynx1, isoform a, amino acids 23-95 for lynx1, isoform b, and amino acids 23-92 for lynx1, isoform c. This corresponds to amino acids 21-92 in the mouse lynx1 gene. Nucleic acids encoding the foregoing polypeptides are also provided, see below. The “lynx polypeptide” or “polypeptide of the invention” as used herein, refers to the above polypeptides or proteins products of the genes comprising the sequences of SEQ ID 8 through SEQ ID 19 and include derivatives thereof, which emcompass variants, fragments, conjugates, multimers, point mutants and fusion proteins thereof, whereby fragments are defined above. There compositions can display one or more functional activities associated with modulatory activity on nAChRs, or binding capabilities on nAChRs. lynx compositions encompass the category of lynx proteins and lynx polypeptides, and also encompass the category of lynx DNAs and RNAs.

Such activities or functionalities may be the polypeptides, original, natural or wild-type activities, or they may be designed and/or engineered. Such design and/or engineering may be achieved, for example, either by deleting amino acids, or adding amino acids to, parts of one, any, both, several, or all of the polypeptides, by fusing polypeptides of different proteins or protein complexes, by adding or deleting post-translational modifications, by adding chemical modifications or appendixes, or by introducing any other mutations or modification by any methods known in the art to this end as set forth in detail below.

The compositions may consist essentially of the polypeptides of a complex, and fragments, analogs, and derivatives thereof. Alternatively, the polypeptides and fragments and derivatives thereof may be a component of a composition that comprises other components, for example, a diluent, such as saline, a pharmaceutically acceptable carrier or excipient, etc.

Polypeptide Derivatives and Analogs

Derivatives or analogs of polypeptides include those molecules comprising regions that are substantially homologous to a polypeptide or fragment thereof (e.g., in various embodiments, at least 40% or 50% or 60% or 70% or 80% or 90% or 95% identity over an amino acid or nucleic acid sequence of identical size or when compared to an aligned sequence in which the alignment is done, for example, by a computer homology program known in the art) or whose encoding nucleic acid is capable of hybridizing to a coding gene sequence, under high stringency, moderate stringency, or low stringency conditions.

Further, one or more amino acid residues within the sequence can be substituted by another amino acid of a similar polarity that acts as a functional equivalent, resulting in a silent alteration. Substitutions for an amino acid within the sequence may be selected from other members of the class to which the amino acid belongs. For example, the nonpolar (hydrophobic) amino acids include alanine, leucine, isoleucine, valine, proline, phenylalanine, tryptophane and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are generally understood to be conservative substitutions.

The derivatives and analogs of the polypeptides of the complex to be stabilized by application of the instant invention can be produced by various methods known in the art. The manipulations that result in their production can occur at the gene or polypeptide level. For example, a cloned gene sequence can be modified by any of numerous strategies known in the art.

Chimeric polypeptides can be made comprising one or several of the polypeptides of a complex to be stabilized by the instant invention, or fragment, derivative, analog thereof (preferably consisting of at least a domain of a protein complex to be stabilized, or at least 6, and preferably at least 10 amino acids of the polypeptide) joined at its amino- or carboxy-terminus via a peptide bond to an amino acid sequence of a different protein.

Such a chimeric polypeptide can be produced by any known method, including: recombinant expression of a nucleic acid encoding the polypeptide (comprising a polypeptide coding sequence joined in-frame to a coding sequence for a different polypeptide); ligating the appropriate nucleic acid sequences encoding the desired amino acid sequences to each other in the proper coding frame, and expressing the chimeric product; and protein synthetic techniques, for example, by use of a peptide synthesizer.

### Manipulations of a Polypeptide Sequence at the Protein Level.

Included within the scope of the invention are polypeptides, polypeptide fragments, or other derivatives or analogs, which are differentially modified during or after translation or synthesis, for example, by glycosylation, acetylation, phosphorylation, amidation, derivatization by known protecting/blocking groups, proteolytic cleavage, etc.

Any of numerous chemical modifications may be carried out by known techniques, including but not limited to specific chemical cleavage by cyanogen bromide, trypsin, chymotrypsin, papain, V8 protease, NaBH.sub.4, acetylation, formylation, oxidation, reduction, metabolic synthesis in the presence of tunicamycin, etc.

In addition, polypeptides, polypeptide fragments, or other derivatives or analogs that can be stabilized using the methods of the instant invention can be chemically synthesized. For example, a polypeptide corresponding to the mature polypeptide can be synthesized by use of a peptide synthesizer. Furthermore, if desired, non-classical amino acids or chemical amino acid analogs can be introduced as substitutions and/or additions into the sequence of one, any, both, several or all of the polypeptides of the complex.

Non-classical amino acids include, but are not limited to, the D-isomers of the common amino acids, fluoro-amino acids, designer amino acids such as beta-methyl amino acids, C gamma-methyl amino acids, N gamma-methyl amino acids, and amino acid analogs in general.

Examples of non-classical amino acids include: alpha-aminocaprylic acid, Acpa; (S)-2-aminoethyl-L-cysteine.HCl, Aecys; aminophenylacetate, Afa; 6-amino hexanoic acid, Ahx; gamma-amino isobutyric acid and alpha-aminoisobytyric acid, Aiba; alloisoleucine, Aile; L-allylglycine, Alg; 2-amino butyric acid, 4-aminobutyric acid, and alpha-aminobutyric acid, Aba; p-aminophenylalanine, Aphe; b-alanine, Bal; p-bromophenylalaine, Brphe; cyclohexylalanine, Cha; citrulline, Cit; beta-chloroalanine, Clala; cycloleucine, Cle; p-cholorphenylalanine, Clphe; cysteic acid, Cya; 2,4-diaminobutyric acid, Dab; 3-amino propionic acid and 2,3-diaminopropionic acid, Dap; 3,4-dehydroproline, Dhp; 3,4-dihydroxylphenylalanine, Dhphe; p-fluorophenylalanine, Fphe; D-glucoseaminic acid, Gaa; homoarginine, Hag; delta-hydroxylysine.HCl, Hlys; DL beta-hydroxynorvaline, Hnyl; homoglutamine, Hog; homophenylalanine, Hoph; homoserine, Hos; hydroxyproline, Hpr; p-iodophenylalanine, Iphe; isoserine, Ise; alpha-methylleucine, Mle; DL-methionine-5-methylsulfoniumchloide, Msmet; 3-(1-naphthyl) alanine, 1Nala; 3-(2-naphthyl)alanine, 2Nala; norleucine, Nle; N-methylalanine, Nmala; Norvaline, Nva; O-benzylserine, Obser; O-benzyltyrosine, Obtyr; O-ethyltyrosine, Oetyr; O-methylserine, Omser; O-methylthreonine, Omthr; O-methyltyrosine, Omtyr; Ornithine, Orn; phenylglycine; penicillamine, Pen; pyroglutamic acid, Pga; pipecolic acid, Pip; sarcosine, Sar; t-butylglycine; t-butylalanine; 3,3,3-trifluoroalanine, Tfa; 6-hydroxydopa, Thphe; L-vinylglycine, Vig; (−)-(2R)-2-amino-3-(2-aminoethylsulfonyl) propanoic acid dihydroxochloride, Aaspa; (2S)-2-amino-9-hydroxy-4,7-dioxanonanoic acid, Ahdna; (2S)-2-amino-6-hydroxy-4-oxahexanoic acid, Ahoha; (−)-(2R)-2-amino-3-(2-hydroxyethylsulfonyl)propanoic acid, Ahsopa; (−)-(2R)-2-amino-3-(2-hydroxyethylsulfanyl)propanoic acid, Ahspa; (2S)-2-amino-12-hydroxy-4,7,10-trioxadodecanoic acid, Ahtda; (2S)-2,9-diamino-4,7-dioxanonanoic acid, Dadna; (2S)-2,12-diamino-4,7,10-trioxadodecanoic acid, Datda; (S)-5,5-difluoronorleucine, Dfnl; (S)-4,4-difluoronorvaline, Dfnv; (3R)-1-1-dioxo-[1,4]thiaziane-3-carboxylic acid, Dtca; (S)-4,4,5,5,6,6,6-heptafluoronorleucine, Hfnl; (S)-5,5,6,6,6-pentafluoronorleucine, Pfnl; (S)-4,4,5,5,5-pentafluoronorvaline, Pfnv; and (3R)-1,4-thiazinane-3-carboxylic acid, Tca. Furthermore, the amino acid can be D (dextrorotary) or L (levorotary). For a review of classical and non-classical amino acids, see Sandberg et al. (Sandberg M. et al., 1998. J. Med. Chem., 41, 2481-2491 incorporated by reference herein).

### Molecular Biological Methods

Nucleic acids encoding one, any, both, several, or all of the polypeptides of complexes that can be stabilized by the methodology of instant invention are provided. The polypeptides, their derivatives, analogs, and/or chimers, of the complex can be made by expressing the DNA sequences that encode them in vitro or in vivo by any known method in the art. Nucleic acids encoding one, any, both, several, or all of the derivatives, analogs, and/or chimers of the complex to be stabilized by the methodology of the instant invention can be made by altering the nucleic acid sequence encoding the polypeptide or polypeptides by substitutions, additions (e.g., insertions) or deletions that provide for functionally active molecules. The sequences can be cleaved at appropriate sites with restriction endonuclease(s), followed by further enzymatic modification if desired, isolated, and ligated in vivo or in vitro. Additionally, a nucleic acid sequence can be mutated in vitro or in vivo, to create and/or destroy translation, initiation, and/or termination sequences, or to create variations in coding regions and/or to form new, or destroy preexisting, restriction endonuclease sites to facilitate further in vitro modification.

Due to the degeneracy of nucleotide coding sequences, many different nucleic acid sequences which encode substantially the same amino acid sequence as one, any, both, several, or all of the polypeptides of complex to be stabilized may be used in the practice of the present invention. These can include nucleotide sequences comprising all or portions of a domain which is altered by the substitution of different codons that encode the same amino acid, or a functionally equivalent amino acid residue within the sequence, thus producing a “silent” (functionally or phenotypically irrelevant) change.

Any technique for mutagenesis known in the art can be used, including but not limited to, chemical mutagenesis, in vitro site-directed mutagenesis, using, for example, the QuikChange Site-Directed Mutatgenesis Kit (Stratagene), etc.

### Nucleic Acids of the Invention

Nucleic Acids of the Invention encode polypeptides of the invention, as defined above, and may be isolated from any source of biological material, including prokaryotic, eukaryotic, mono-cellular, multi-cellular, animal, plant, fungus, vertebrate, invertebrate, mammalian, human, avian, insect, nematode, simian, monkey, murine, rat, mouse, porcine, bovine, feline, equine, canine, fruit fly, or firefly animals, and any other species or biological being. Nucleic acids of the invention include fragments, analogs, derivatives, and fusions analogous to the definitions above for polypeptides of the invention.

DNA compositions of the invention comprise DNA molecules of the nucleic acids of the invention, and may be obtained by standard procedures known in the art from cloned DNA (e.g., a DNA “library”), by chemical synthesis, by cDNA cloning, by the cloning of genomic DNA, or fragments thereof, purified from the desired cell (see e.g., Sambrook et al., 1985, Glover (ed.). MRL Press, Ltd., Oxford, U.K, vol. I, II. incorporated by reference herein). The DNA may also be obtained by reverse transcribing cellular RNA, prepared by any of the methods known in the art, such as random- or poly A-primed reverse transcription. Such DNA may be amplified using any of the methods known in the art, including PCR and 5′RACE techniques (Weis et al., 1992, Trends Genet., 8, 263-264, Frohman, 1994, PCR Methods Appl., 4, S40-58, all incorporated by reference herein).

Whatever the source, the gene should be molecularly cloned into a suitable vector for propagation of the gene and/or administration of the composition to individuals for therapeutic purposes, as described herein. Additionally, the DNA may be cleaved at specific sites using various restriction enzymes, DNAse may be used in the presence of manganese, or the DNA can be physically sheared, as for example, by sonication. The linear DNA fragments can then be separated according to size by standard techniques, such as agarose and polyacrylamide gel electrophoresis and column chromatography.

Cloning

Once the DNA fragments are generated, identification of the specific DNA fragment containing the desired gene may be accomplished in a number of ways. For example, clones can be isolated by using PCR techniques that may either use two oligonucleotides specific for the desired sequence, or a single oligonucleotide specific for the desired sequence, using, for example, the 5′ RACE system (Cale et al., 1998, Methods Mol. Biol., 105, 351-371, Frohman, 1994 PCR Methods Appl., 4, S40-58, all incorporated by reference herein). The oligonucleotides may or may not contain degenerate nucleotide residues. Alternatively, if a portion of a gene or its specific RNA or a fragment thereof is available and can be purified and labeled, the generated DNA fragments may be screened by nucleic acid hybridization to the labeled probe (e.g. Benton and Davis, 1977, Science, 196, pp. 180-182, incorporated by reference herein). Those DNA fragments with substantial homology to the probe will hybridize. It is also possible to identify the appropriate fragment by restriction enzyme digestion(s) and comparison of fragment sizes with those expected according to a known restriction map if such is available. Further selection can be carried out on the basis of the properties of the gene.

The presence of the desired gene may also be detected by assays based on the physical, chemical, or immunological properties of its expressed product. For example, cDNA clones, or DNA clones which hybrid-select the proper mRNAs, can be selected and expressed to produce a polypeptide that has, for example, similar or identical electrophoretic migration, isoelectric focusing behavior, proteolytic digestion maps, hormonal or other biological activity, binding activity, enzymatic activity, or antigenic properties as known for a protein.

Using an antibody to a known protein, other proteins may be identified by binding of the antibody labeled by any means known to one of ordinary skill in the art to expressed putative proteins, for example, in an ELISA (enzyme-linked immunosorbent assay)-type procedure; alternatively, an unlabeled antibody in conjunction with a labeled secondary antibody for sandwiched detection. Further, using a binding protein specific to a known protein, other proteins may be identified by binding to such a protein either in vitro or a suitable cell system, such as the yeast-two-hybrid system (see e.g. Clemmons, Mol. Reprod. Dev., 1993, 35, 368-374, Loddick et al., 1998, Proc. Natl. Acad. Sci., U.S.A., 95, 1894-1898, all incorporated by reference herein).

A gene can also be identified by mRNA selection using nucleic acid hybridization followed by in vitro translation. In this procedure, fragments are used to isolate complementary mRNAs by hybridization. Such DNA fragments may represent available, purified DNA of another species (e.g., Drosophila, mouse, human). Immunoprecipitation analysis or functional assays (e.g. aggregation ability in vitro, binding to receptor, etc.) of the in vitro translation products of the isolated products of the isolated mRNAs identifies the mRNA and, therefore, the complementary DNA fragments that contain the desired sequences.

In addition, specific mRNAs may be selected by adsorption of polysomes isolated from cells to immobilized antibodies specifically directed against protein. A radiolabeled cDNA can be synthesized using the selected mRNA (from the adsorbed polysomes) as a template. The radiolabeled mRNA or cDNA may then be used as a probe to identify the DNA fragments from among other genomic DNA fragments.

Alternatives to isolating the genomic DNA include, chemically synthesizing the gene sequence itself from a known sequence or making cDNA to the mRNA which encodes the polypeptide. For example, RNA for cDNA cloning of the gene can be isolated from cells that express the gene.

RNA compositions of the invention are transcripts of the above describe DNA compositions of the invention, including, fragments, analogs, derivatives, and fusions thereof.

### Potential Applications of the Invention

Conditions, disorder, and disease that may be treated with polypeptides of the invention include neurological disorder, and encompass pain, neuropathic pain, schizophrenia, cognitive impairments, dementias, including Alzheimer's disease, and Parkinson's disease and encompasses mood disorders, anxiety disorders and depressive disorders. It can be also used to treat ADNFLE, a type of epilepsy, which is caused by a mutation in nAChRs.

### Obtaining Polypeptides

### Purification of Polypeptides

One, any, several or all of the polypeptides of the instant invention may be obtained by any protein purification methods known in the art. Such methods include, but are not limited to, chromatography (e.g. ion exchange, affinity, and/or sizing column chromatography), ammonium sulfate precipitation, centrifugation, differential solubility, or by any other standard technique for the purification of proteins. The polypeptides may be purified from any source that produces one, any, both, several or all of the polypeptides of a complex of the desired complex to be stabilized. For example, polypeptides may be purified from sources including, prokaryotic, eukaryotic, mono-cellular, multi-cellular, animal, plant, fungus, vertebrate, mammalian, human, porcine, bovine, feline, equine, canine, avian, tissue culture cells, and any other natural, modified, engineered, or any otherwise not naturally occurring source. For a review of purification techniques, see Protein Purification Protocols (Methods in Molecular Biology), Cutler, (ed.), Humana press, 2003, incorporated by reference herein).

### Expression of DNA Encoding the Polypeptides of the Complex

Expression and Cloning Vectors

Identified and isolated nucleic acids of the invention can then be inserted into an appropriate cloning or expression vector. A large number of vector-host systems known in the art may be used. Possible vectors include plasmids or modified viruses, but the vector system must be compatible with the host cell used. Such vectors include bacteriophages such as lambda derivatives, or plasmids such as PBR322 or pUC plasmid derivatives or the Bluescript vector (Stratagene).

The insertion into a cloning vector can, for example, be accomplished by ligating the DNA fragment into a cloning vector that has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Furthermore, the gene and/or the vector may be amplified using PCR techniques and oligonucleotides specific for the termini of the gene and/or the vector that contain additional nucleotides that provide the desired complementary cohesive termini. In alternative methods, the cleaved vector and a gene may be modified by homopolymeric tailing (Cale et al., 1998, Methods Mol. Biol., 105, 351-371, incorporated by reference herein).

Recombinant molecules can be introduced into host cells via transformation, transfection, infection, electroporation, etc., so that many copies of the gene sequence are generated.

Preparation of DNA

In specific embodiments, transformation of host cells with recombinant DNA molecules that incorporate an isolated gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The sequences provided by the instant invention include those nucleotide sequences encoding substantially the same amino acid sequences as found in native polypeptides, and those encoded amino acid sequences with functionally equivalent amino acids, as well as those encoding other derivatives or analogs, as described below for derivatives and analogs.

Structure of Genes and Polypeptides

The amino acid sequence of a polypeptide can be derived by deduction from the DNA sequence, or alternatively, by direct sequencing of the polypeptide, for example, with an automated amino acid sequencer.

A polypeptide sequence can be further characterized by a hydrophilicity analysis (Hopp and Woods, 1981, Proc. Natl. Acad. Sci., U.S.A., 78, 3824, incorporated by reference herein). A hydrophilicity profile can be used to identify the hydrophobic and hydrophilic regions of the polypeptide and the corresponding regions of the gene sequence which encode such regions.

Secondary, structural analysis (Chou and Fasman, 1974, Biochem., 13, 222-245, incorporated by reference herein) can also be done, to identify regions of a polypeptide that assume specific secondary structures. Manipulation, translation, and secondary structure prediction, open reading frame prediction and plotting, as well as determination of sequence homologies, can also be accomplished using computer software programs available in the art. Other methods of structural analysis include X-ray crystallography, nuclear magnetic resonance spectroscopy and computer modeling.

### DNA Vectors Constructs

The nucleotide sequence coding for one, any, several or all of the polypeptides, or functionally active analogs or fragments or other derivatives thereof, can be inserted into an appropriate expansion or expression vectors, i.e., a vector which contains the necessary elements for the transcription alone, or transcription and translation, of the inserted protein-coding sequence(s). The native genes and/or their flanking sequences can also supply the necessary transcriptional and/or translational signals. Expression of a nucleic acid sequence encoding a polypeptide or peptide fragment may be regulated by a second nucleic acid sequence so that the polypeptide is expressed in a host transformed with the recombinant DNA molecule. For example, expression of a polypeptide may be controlled by any promoter/enhancer element known in the art.

Promoters which may be used to control gene expression include, as examples, the SV40 early promoter region, the promoter contained in the 3′ long terminal repeat of Rous sarcoma, the herpes thymidine kinase promoter, the regulatory sequences of the metallothionein gene; prokaryotic expression vectors such as the beta-lactamase promoter, or the lac promoter; plant expression vectors comprising the nopaline synthetase promoter or the cauliflower mosaic virus 35S RNA promoter, and the promoter of the photosynthetic enzyme ribulose biphosphate carboxylase; promoter elements from yeast or other fungi such as the Gal 4 promoter, the alcohol dehydrogenase promoter, phosphoglycerol kinase promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell, 38, 639-646) a gene control region which is active in pancreatic beta cells (Hanahan, 1985, Nature, 315, 115-122), an immunoglobulin gene control region which is active in lymphoid cells (Grosschedl et al., 1984, Cell, 38, 647-658), mouse mammary tumor virus control region which is active in testicular, breast, lymphoid and mast cells (Leder et al., 1986, Cell, 45, 485-495), albumin gene control region which is active in liver (Pinkert et al., 1987, Genes Dev., 1, 268-276), alpha-fetoprotein gene control region which is active in liver (Krumlauf et al., 1985, Mol. Cell. Biol., 5, 1639-1648), alpha 1-antitrypsin gene control region which is active in the liver (Kelsey et al., 1987, Genes Dev., 1, 161-171), beta-globin gene control region which is active in myeloid cells (Magram et al., 1985, Nature, 315, 338-340); myelin basic protein gene control region which is active in oligodendrocyte cells in the brain (Readhead et al., 1987, Cell, 48, 703-712), myosin light chain-2 gene control region which is active in skeletal muscle (Shani, Nature, 1985, 314, 283-286), and gonadotropic releasing hormone gene control region which is active in the hypothalamus (Mason et al., 1986, Science, 234, 1372-1378).

In a specific embodiment, a vector is used that comprises a promoter operably linked to a gene nucleic acid, one or more origins of replication, and, optionally, one or more selectable markers (e.g., an antibiotic resistance gene). In bacteria, the expression system may comprise the lac-response system for selection of bacteria that contain the vector. Expression constructs can be made, for example, by subcloning a coding sequence into one the restriction sites of each or any of the pGEX vectors (Pharmacia, Smith and Johnson, 1988, Gene, 67, 3140). This allows for the expression of the protein product.

Vectors containing gene inserts can be identified by three general approaches: (a) identification of specific one or several attributes of the DNA itself, such as, for example, fragment lengths yielded by restriction endonuclease treatment, direct sequencing, PCR, or nucleic acid hybridization; (b) presence or absence of “marker” gene functions; and, where the vector is an expression vector, (c) expression of inserted sequences. In the first approach, the presence of a gene inserted in a vector can be detected, for example, by sequencing, PCR or nucleic acid hybridization using probes comprising sequences that are homologous to an inserted gene. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene in the vector. For example, if the gene is inserted within the marker gene sequence of the vector, recombinants containing the insert an identified by the absence of the marker gene function. In the third approach, recombinant expression vectors can be identified by assaying the product expressed by the recombinant expression vectors containing the inserted sequences. Such assays can be based, for example, on the physical or functional properties of the polypeptide in in vitro assay systems, for example, binding with anti-protein antibody.

Once a particular recombinant DNA molecule is identified and isolated, several methods known in the art may be used to propagate it. Once a suitable host system and growth conditions are established, recombinant expression vectors can be propagated and prepared in quantity. Some of the expression vectors that can be used include human or animal viruses such as vaccinia virus or adenovirus; insect viruses such as baculovirus; yeast vectors; bacteriophage vectors (e.g., lambda phage), and plasmid and cosmid DNA vectors.

Once a recombinant vector that directs the expression of a desired sequence is identified, the gene product can be analyzed. This is achieved by assays based on the physical or functional properties of the product, including radioactive labeling of the product followed by analysis by gel electrophoresis, immunoassay, etc.

### Systems of Gene Expression and Protein Purification

A variety of host-vector systems may be utilized to express the protein-coding sequences. These include, as examples, mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

In a specific embodiment, the gene may be expressed in bacteria that are protease deficient, and that have low constitutive levels and high induced levels of expression where an expression vector is used that is inducible, for example, by the addition of IPTG to the medium.

In yet another specific embodiment, one, any, several or all of the polypeptides of the invention may be expressed with signal peptides, such as, for example, pelB bacterial signal peptide, that directs the polypeptide to the bacterial periplasm (Lei et al. J. Bacteriol., 1977, 169, 437, incorporated by reference herein). Alternatively, polypeptide may be allowed to form inclusion bodies, and subsequently be resolubilized and refolded (Kim et al., 1997, Mol. Immunol, 34, 891, incorporated by reference herein). Any of the methods previously described for the insertion of DNA fragments into a vector may be used to construct expression vectors containing a chimeric gene consisting of appropriate transcriptional/translational control signals and the protein coding sequences. These methods may include in vitro recombinant DNA and synthetic techniques and in vivo recombinants (genetic recombination).

In addition, a host cell strain may be chosen that modulates the expression of the inserted sequences, or modifies and processes the gene product in the specific fashion desired. Expression from certain promoters can be elevated in the presence of certain inducers; thus, expression of the genetically engineered polypeptides may be controlled. Furthermore, different host cells have characteristic and specific mechanisms for the translational and post-translational processing and modification (e.g., glycosylation, phosphorylation of proteins. Appropriate cell lines or host systems can be chosen to ensure the desired modification and processing of the foreign polypeptide(s) expressed. For example, expression in a bacterial system can be used to produce a non-glycosylated core protein product. Expression in yeast may produce a glycosylated product. Expression in mammalian cells can be used to attain “native” glycosylation of a heterologous polypeptide. Furthermore, different vector/host expression systems may effect processing reactions to different extents.

In other embodiments of the invention, one, any, several or all of the polypeptides of the invention, and/or fragments, analogs, or derivative(s) thereof may be expressed as a fusion-, or chimeric, protein product (comprising the polypeptide, fragment, analog, or derivative joined via a peptide bond to a heterologous polypeptide sequence of a different protein). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, for example, by use of a peptide synthesizer.

### Assaying Compositions of the Invention

Compositions of the invention can be assayed as described in the following.

Protein Binding

Proteins or polypeptides of the invention can be assayed through a number of binding assays which assess protein-protein interactions. The polypeptide can exist in a number of forms including labeled with an antigen tag, fluorescent tag, an unnatural amino acid, crosslinked to another protein, covalently bound to another protein or peptide etc, The assays can include but are not limited to ELISA, FRET binding and imaging (Chan et al, 2001, Cytometry, 44, 361-368, incorporated by reference herein), protein chip microarray assay (Hu et al., 2007, Proteomics, 7, 2151-2161, incorporated by reference herein), protein pull down assay (Lin et al., 2005, Toxicon. 47, 265-270, incorporated by reference herein), cellular binding of labeled polypeptides on cells or neurons (Sekine-Aizawa and Huganir, 2004, Proc. Natl. Acad. Sci., 101, 17114-17119, incorporated by reference herein), competition with labeled alpha-btx or other proteins (Stiles, 1993, Toxicon, 31, 825-834, incorporated by reference herein), TIRF imaging (Chung et al., 2007, Biophys J., 93, 1747-1757, incorporated by reference herein), far western, and cross-linking experiments, and toxin binding assays. (Salminent al., 2005, Neuropharmacol., 48, 696-705, incorporated by reference herein).

Functional/Electrophysiological Assays:

The function of the compositions of the invention can be tested by incubating the lynx composition with brain and/or neuronal tissue and/or cells expressing recombinant nAChRs. Neuronal tissue can include whole brain assays, brain slices in vitro, primary neuronal cultures, and heterologously expressed receptors in cells, where the cell type is, for example, but not limited to, xenopus ooctyes or mammalian cells. To test for functional activity of the polypeptide of the invention, electrophysiological assays include the following: (1) the lynx composition can be incubated with nAChR expression cells to assess its functional properties. These cells can include but are not limited to mammalian cells, neurons, or cells from other species, transfected with nAChr cDNA, cRNA, RNA or BAC DNA. The assays systems can include activity on neurons or cells which express heterologous nAChRs or natively expressed nAChRs; they can also include oocytes, such as from xenopus, injected with RNA, DNA or cDNA.

Patch clamp experiments include the following: inside-out and outside-out patch, perforated patch, intact patch recording, and single channel recording (Hille, B. Ion Channels of Excitable Membranes, Sinauer Associates, 1992, and Hamill et al, 1981, Pflugers Arch., 391, 85-100, all incorporated by reference herein), and planar patch electrode (Li, et al., 2006, Nano Lett. 6, 815-819, incorporated by reference herein). The activity of the polypeptide of the invention can also be testing through in vivo recordings, including single unit recording, sharp electrode and microelectrode recordings of spontaneous and evoked responses (Kandel et al., 2000, Principles of Neural Science, 4th ed., McGraw-Hill, New York, Modern Techniques in Neuroscience Research, Windhorst and Johansson, (Eds), Springer Lab Manuals, 146-155, all incorporated by reference herein). Functional assays can also include using measurements in brain slices (Modern Techniques in Neuroscience Research, Windhorst and Johansson, (Eds), Springer Lab Manuals, 311-318, incorporated by reference herein) to measure action potential frequency, evoked responses and field potential recordings, action potential frequency and SPC measurements (Modern Techniques in Neuroscience Research, Windhorst and Johansson, (Eds), Springer Lab Manuals, 134-146, incorporated by reference herein), in addition to multielectrode recordings (Steidl et al., 2006, Brain Res., 1096, 70-84, incorporated by reference herein).

Assays to measure NT levels in response to infusion or application of composition of the invention include microdialysis in the brain, (Ding et al., 2007, Neurosci Lett. 422, 175-180, incorporated by reference herein), and Rb efflux measurements from synptosome preparations (Nashmi et al. 2003, J. Neurosci. 23, 11554-11567, Gill et al., 2007, Assay Drug Dev. Technol., 5, 373-80, all incorporated by reference herein).

The effects of the lynx polypeptide of the invention can be studied by optical imaging on neurons, cellular fragments, or heterologously expressing receptors in cells, using voltage and/or membrane sensitive dyes (Vijayaraghavan et al., 1992, Neuron, 8, 353-362, incorporated by reference herein, or using intrinsic signals (Wang et al., 2007, Neurosci Lett., 2, 133-138, incorporated by reference herein), also recordings of whole tissues such as EEG, EMG, EKG electrocardiogram.

The effect of the composition of the invention can include change in the agonist sensitivity profile to agonists including but not limited to nicotine, acetylcholine, epibatidine, galantamine, etc. and can include but are not limited to, changes in EC50, change in maximal response, change in Hill coefficient, change in stoichiometry, change in receptor levels, change in functional assembly, desensitization kinetice, recovery from desensitization, alterations in NT release, change in IPSC frequency, EPSC frequency, AP frequency, membrane potential, bursting pattern, mean open time, amplitude of single channel open events.

The polypeptide of the invention can also be assessed using behavioral assays which include but are not limited to: motor assays, including open field and rotarod assays, learning and memory tests, such as water maze, fear conditioning and passive avoidance assays, anxiety tests, such as elevated mazes, light-dark box, social interaction tests, pain sensitivity assays, such as hot-plate and tail flick tests, (Crawley, J. N., 2007, What's Wrong With My Mouse? Behavioral Phenotyping of Transgenic and Knockout Mice. Second Edition. John Wiley & Sons, Hoboken N.J., incorporated by reference herein)

### Therapeutic Uses

The present invention is directed to therapies which involve administering compositions of the invention to an animal, preferably a mammal, and most preferably a human, patient for treating one or more of the disclosed diseases, disorders, or conditions. Therapeutic compounds of the invention include, but are not limited to, polypeptides of the invention (including fragments, variants analogs, fusions, and derivatives thereof as described herein) and nucleic acids encoding polypeptides of the invention (including fragments, analogs derivatives, and fusions thereof). The compostions of the invention can be used to treat diseases, disorders or conditions associated with aberrant expression and/or activity of a polypeptide of the invention; alternatively, the compostions of the invention can be used to treat, inhibit or prevent diseases, disorders or conditions associated with aberrant physiology that can be correct by therapeutic application of the compositions of the present invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. compositions of the invention may be provided in pharmaceutically acceptable compositions as known in the art or as described herein.

A summary of the ways in which the compositions of the present invention may be used therapeutically includes administering polypeptides of the present invention locally in the body. With the teachings provided herein, one of ordinary skill in the art will know how to use the compositions of the present invention for diagnostic, monitoring or therapeutic purposes without undue experimentation.

Polypeptides of this invention may be advantageously utilized in combination with other therapeutic molecules, such as with lymphokines or hematopoietic growth factors, or small molecule therapeutics useful in the treatment of the diseases, disorders or conditions that may be addressed.

The polypeptides of the invention may be administered alone or in combination with other types of treatments. Generally, administration of products of a species origin or species reactivity that is the same species as that of the patient is preferred. Thus, in a preferred embodiment, human polypeptides or nucleic acids of the invention, including fragments, variants, derivatives, or analogs thereof, are administered to a human patient for therapy or prophylaxis.

In one embodiment, this invention provides a pharmaceutical composition comprising an effective amount of an composition of the invention, and a pharmaceutically acceptable carrier. As used herein, “an effective amount” means an amount required to achieve a desired end result. The amount required to achieve the desired end result will depend on the nature of the specific composition of the invention, which can be determined as described above without undue experimentation, and the diseases, conditions, or disorders being treated, and can be determined by standard clinical techniques. In addition, in vitro assays may optionally be employed to help identify optimal dosage ranges. The precise dose to be employed will also depend on the route of administration and the seriousness of the disease or disorder, and should be decided according to the judgment of the practitioner and each subject's circumstances. Effective doses may be extrapolated from dose-response curves derived from in vitro or animal model test systems. Various delivery systems are known and can be used to administer a pharmaceutical composition of the present invention. Methods of introduction include delivery directly to the CNS. This includes infusion of compositions of the invention directly into the brain or spinal cord. They also include methods of coating or containing the polypeptide, DNA, or RNA, in the bloodstream, to be delivered to the central nervous system, wherein it is inactive outside of the central nervous system, but active when delivered to the CNS.

The compounds of the invention are administered to the CNS by any convenient route, for example by infusion., and may be administered together with other biologically active agents. Administration can be systemic, whereby the composition is targeted to the CNS, or local infusion, for example, during surgery, by injection, by means of a catheter, or by means of an implant, said implant being of a porous, non-porous, or gelatinous material, including membranes, such as silastic membranes, or fibers. Pharmaceutical compositions of the invention may be administered into the central nervous system by any suitable route, including, for example, but not limited to, intraventricular and intrathecal injection; intraventricular injection may be facilitated by an intraventricular catheter, for example, attached to a reservoir, such as an Ommaya reservoir. Pulmonary administration may also be employed, for example, but not limited to, by use of an inhaler or nebulizer, and formulation with an aerosolizing agent.

In one embodiment, a pump may be used (see Langer, supra and Sefton, 1987, CRC Crit. Ref. Biomed. Eng. 14, 201, Buchwald et al., 1980, Surgery 88, 507, Saudek et al., 1989. N. Engl. J. Med., 321, 574, all incorporated by reference herein). In another embodiment, polymeric materials can be used (see Medical Applications of Controlled Release, Langer and Wise (eds.), 1974, CRC Pres., Boca Raton, Fla., Controlled Drug Bioavailability, 1984, Drug Product Design and Performance, Smolen and Ball (eds.), Wiley, New York, Ranger and Peppas, 1983, Macromol. Sci. Rev. Macromol. Chem. 23, 61, see also Levy et al., 1985, Science, 228, 190, During et al, 1989, Ann. Neurol., 25, 351, Howard et al., 1989, J. Neurosurg. 71, 105, all incorporated by reference herein). In yet another embodiment, a controlled release system can be placed in proximity of the therapeutic target, i.e., the brain, thus requiring only a fraction of the systemic dose (see, e.g., Goodson, 1984. in Medical Applications of Controlled Release, vol. 2, 115-138, incorporated by reference herein).

Other controlled release systems are discussed in the review by Langer (Langer, 1990, Science, vol. 249, 527-1533, incorporated by reference).

In a preferred embodiment, the polypeptide of the present invention is formulated in accordance with routine procedures as a pharmaceutical composition adapted for intravenous administration to human beings. Typically, compositions for administration are solutions in sterile isotonic aqueous buffer. Where necessary, the compositions of the present invention may also include a solubilizing agent and a local anesthetic. Generally, the ingredients are supplied either separately or mixed together in unit dosage form, for example, as a dry lyophilized powder or water free concentrate in a hermetically sealed container such as an ampoule or sachette indicating the quantity of active agent. Where the composition of the present invention is to be administered by infusion, it can be dispensed with an infusion bottle containing sterile pharmaceutical grade water or saline. Where the polypeptide of the present invention is administered by injection, an ampoule of sterile water for injection or saline can be provided so that the ingredients may be mixed prior to administration.

### Considerations for Pharmaceutical Compositions

Polypeptides of the Invention

Polypeptides of the invention should be administered in a carrier that is pharmaceutically acceptable. The term “pharmaceutically acceptable” means approved by a regulatory agency of the Federal or a state government or listed in the U.S. Pharmacopeia or other generally recognized pharmacopeia or receiving specific or individual approval from one or more generally recognized regulatory agencies for use in animals, and more particularly in humans. The term “carrier” refers to a diluent, adjuvant, excipient, or vehicle with which the therapeutic is administered. Such pharmaceutical carriers can be sterile liquids, such as water, organic solvents, such as certain alcohols, and oils, including those of petroleum, animal, vegetable or synthetic origin, such as peanut oil, soybean oil, mineral oil, sesame oil and the like. Buffered saline is a preferred carrier when the pharmaceutical composition is administered intravenously. Saline solutions and aqueous dextrose and glycerol solutions can also be employed as liquid carriers, particularly for injectable solutions. The composition, if desired, can also contain minor amounts of wetting or emulsifying agents, or pH buffering agents. These compositions can take the form of solutions, suspensions, emulsion and the like. Examples of suitable pharmaceutical carriers are described in “Remington's Pharmaceutical Sciences” by E. W. Martin. Such compositions will contain a therapeutically effective amount of the therapeutic antibody of the invention, preferably in purified form, together with a suitable amount of carrier so as to provide the form for proper administration to the patient. The formulation should suit the mode of administration. In a preferred embodiment, the antibody of the present invention is formulated in accordance with routine procedures as a pharmaceutical composition adapted for intravenous administration to human beings. Typically, compositions for intravenous administration are solutions in sterile isotonic aqueous buffer.

Nucleic Acids of the Invention

Nucleic Acid compositions of the invention may be the administered to the CNS of an individual, by any means known to one of ordinary skill in the art. In one embodiment of the invention, the nucleic acids are taken up by the cells of the CNS of the individual, and drive or inhibit the expression of polypeptides of the invention. An efficient strategy for enhancing nucleic acid delivery in vivo is to protect the nucleic acid from degradation, thereby maintaining the administered nucleic acid at the target site in order to further increase its cellular uptake. In one embodiment of the invention, the effective concentration of the nucleic acid at the cell surface may be increased for in vitro administration in order to enhance the efficiency of uptake/transfection. Formulations of the nucleic acid compositions of the present invention may comprise a compound which protects the nucleic acid and/or prolongs the localized bioavailability of the nucleic acid when administered to an organism in vivo, as described in U.S. Pat. No. 6,514,947, herewith incorporated in its entirety herein by reference.

The use of formulated DNA expression vectors has a significant advantage in that fewer molecules of the vector will be required for a therapeutic effect. Furthermore, the formulated DNA vectors may provide controlled persistence of the therapeutic effect. The use of formulated DNA expression vectors for administration has a significant advantage in that a CNS tissue-specific promoter can be incorporated such that the therapeutic gene product is produced only in the CNS even if the vector is distributed elsewhere, thus restricting the biological effect of the vector to the desired target.

As another example, formulated DNA vectors constructed to direct or inhibit expression of one or more polypeptides of the invention, may be introduced analogously to methods in which it is introduced directly into fluid spaces such that cells associated with the fluid space can incorporate the vector construct and express the recombinant gene, resulting in expression of nucleic acids of the invention in the above tissues, as described in detail in U.S. Pat. No. 5,770,580, which is hereby incorporated in its entirety herein by reference. Such formulated DNA vectors can be used to treat diseases affecting the CNS by delivering vectors that express a therapeutic gene product that is secreted into the circulation of the brain, for instance.

### EXAMPLE 1

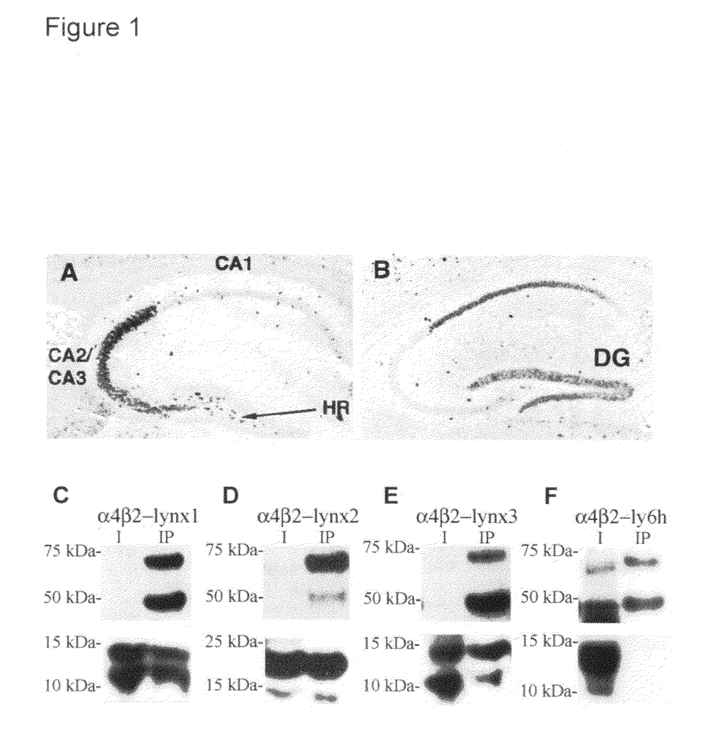

Lynx polypeptides were demonstrated to bind the neuro acetylcholine receptor (hereinafter “nAChR”). FIG. 1 shows the interaction between mouse lynx1, lynx2 lynx3, and ly6H, a lynx-related gene, and the nAChRin co-immunoprecipitation experiments. In FIGS. 1A and B, in situ hybridization experiment demonstrate complementary expression patterns of lynx1 and lynx2 genes: in the hippocampus, lynx1 is expressed in the CA2/CA3 region of the hippocampus, and cells in the hilar region of the dentate gyrus (FIG. 1A), whereas the lynx2 gene is expressed in the CA1 region and to a lesser degree the pyramidaly neurons of the dentate gyrus (FIG. 1B). Figures C-F depict co-immunoprecipitation studies of transiently transfected HEK293 cells with alpha4beta2 nAChRs co-transfected with lynx1 (C), lynx2 (D), lynx3 (E) and ly6H (F), a related family member. Immunoprecipitation experiments were carried out with antibodies to the nAChRs, and the resulting analyte was probed on a Western blot with nAChRs antibodies (upper set of panels) and antibodies to the lynx polypeptides (lower panels); these results indicate stable associations of nAChRs specifically with lynx1, lynx2 and lynx3, but not ly6H.

Modulatory capabilities of lynx polypeptides were revealed in recordings of xenopus oocytes heterologously expressing nAChR and lynx cRNA. lynx1, lynx2 and lynx3 enhance desensitization of ACh-evoked currents mediated through alph4beta2 nAChRs in oocytes. Figure A demonstrates . . . , in that representative recordings of voltage clamped oocytes expressing alph4beta2 nAChRs alone, or in combination with lynx1, lynx2, lynx3 and ly6H. The inward currents were evoked by 20 sec periods of superfusion (horizontal calibration bar) with external saline containing 1 mM ACh. ACh evoked responses in oocytes coexpressing alph4beta2 nAChRs with lynx1, lynx2 or lynx3 showed significantly faster desensitization during agonist application immediately after the initial peak. ly6h had no effect on desensitization when coexpressed with alph4beta2 receptors. B and C). The differences in desensitization are shown with bar graphs. As described in (2), two exponentials equations were fitted to the desensitization currents during ACh application. Using these equations, fast (B) and slow (C) time constants were calculated and the average values of these constants for ACh responses shown. In oocytes coexpressing alph4beta2 nAChRs with lynx1, lynx2 or lynx3, the fast time constant is significantly faster, while the slow time constant during the plateau phase remained the same. Both constants are unaffected in oocytes coexpressing Ly6H.

### EXAMPLE 2

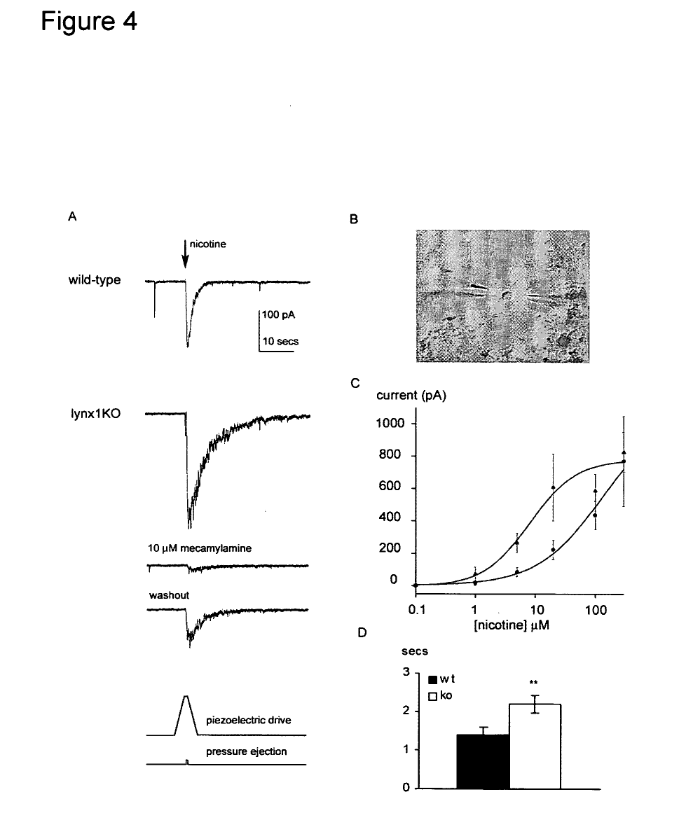

Nicotine-induced currents in lynx1 null mutant mice demonstrate hypersensitivity to agonist. Direct measurements of nicotine sensitivity were carried out using whole-cell patch clamp recordings of neurons in brain slices of lynx1^{-/-} vs. wild-type mice. The medial habenula was chosen for these experiments because of the high level of expression of nAChRs and the co-expression of lynx1 and nAChRs in this region. Application of a 250 ms nicotine pulse to neurons in the medial habenula (FIG. 4B) elicited larger peak responses in slices from lynx1^{-/-} animals than those of wild-type animals (FIG. 4A) for nicotine concentrations between 1 and 20 μM. For the entire tested nicotine concentration range (0.1 μM to 300 μM), the data reveal a decrease in the EC₅₀ from 89.0±2.2 μM in wild-type mice to 9.0±0.7 μM in lynx1^{-/-} mice (FIG. 4C). These currents were blocked by the nAChR antagonist, mecamylamine (mec), and recovered during wash out (middle traces), indicating a specific involvement of nAChRs in this response. Analysis of the decay rate of the nicotine response revealed that the half-maximal response times in neurons from lynx1^{-/-} mice (2.2±0.22 s) were significantly prolonged compared to those of wild-type neurons, (1.4±0.19 s) (FIG. 4D). The peak responses are determined primarily by activation processes and show clear hypersensitivity; and these kinetic data also suggest that removal of lynx1 can alter the deactivation and/or desensitization processes of nAChRs in vivo.

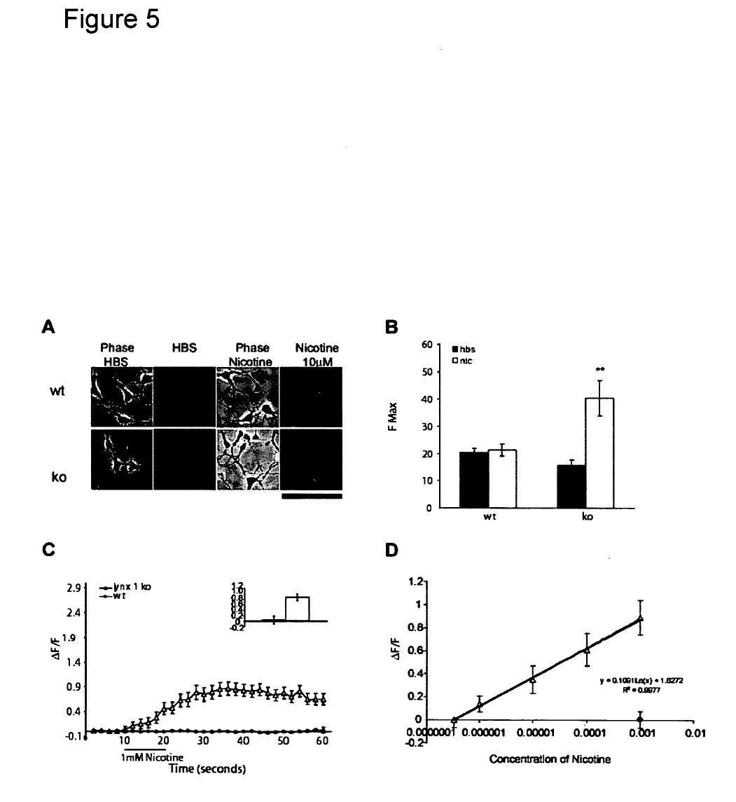

lynx1 null mutant neurons display increased sensitivity to nicotine. Since some aspects of nicotinic receptor hypersensitivity may be mediated via intracellular Ca²⁺ levels, the effect of nicotine on Ca²⁺ levels in primary cortical cultures from lynx1^{-/-} and wild-type mice were measured. FIG. 5 shows neurons which were exposed either to buffer, or 10 μM nicotine. Cultured cells were then loaded with the Ca²⁺ sensitive indicator fluo-3 and fluorescence measurements were obtained (FIG. 5A). Incubation of wild-type cultures with 10 μm nicotine did not result in a significant change in steady state Ca²⁺ levels, whereas lynx1^{-/-} cultures demonstrated a 2-fold increase in fluorescence (FIG. 5B). These data indicate an effect of lynx1 on ligand sensitivity and/or desensitization of nAChRs. Acute responses to nicotine were measured using fluorescence levels in real time. Nicotine elicited a significant increase in Ca⁺ levels in lynx1^{-/-} but not in wild-type cultures (FIG. 5C). Dose-response measurements indicate that ˜1 μM nicotine is sufficient for activation of nAChRs and elevation of Ca²⁺ levels in lynx1^{-/-} cultures (FIG. 5D); but under these conditions no change in fluorescence was observed at any of the concentrations tested for wild-type cultures (FIG. 5D). Altered response properties of nAChRs in lynx1 mutant cells can result in elevated intracellular Ca²⁺ levels, perhaps leading to changes in intracellular signaling.

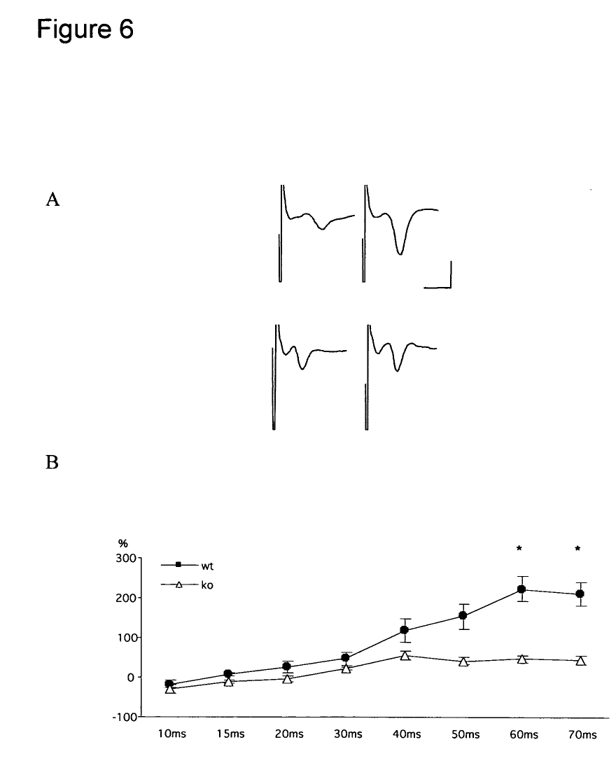

Removal of lynx1 alters synaptic activity. Maintenance of intracellular Ca²⁺ homeostasis is critical for neuronal excitability and synaptic activity. Given the enhanced sensitivity of neurons from lynx1 null mutant mice to nicotine, and the elevations in Ca²⁺ levels observed in these cells in response to nicotine, it seemed likely that changes in synaptic NT release would be present in lynx1 null mutant mice. Since synaptic responses are sensitive to nicotine in the hippocampus and both lynx1 and nAChRs are present in this brain region, excitatory synaptic responses were tested and found to be altered in lynx1^{-/-} hippocampal slices. Field potential recordings of evoked CA3 to CA1 synaptic responses and measured PPF ratios, an indicator of the probability of NT release, by applying two consecutive stimuli at intervals ranging from 10-70 ms. As expected, potentiation of the second response relative to the first response was observed at latency intervals of 30 to 70 ms in wild-type slices (FIG. 4A), as residual Ca²⁺ from the first stimulus adds to the Ca²⁺ influx during the second, leading to more vesicle fusion and a potentiation of NT release. In contrast, PPF ratios in lynx1^{-/-} slices were significantly reduced relative to wild-type responses at intervals of 50-70 ms (FIG. 6A, 6B). Previous studies have suggested that a reduction in PPF reflects an increased probability of vesicle fusion and NT release, leading to a depletion of vesicle pools available to respond to subsequent stimuli. These data suggest alterations in synaptic efficacy in lynx1^{-/-} mice.

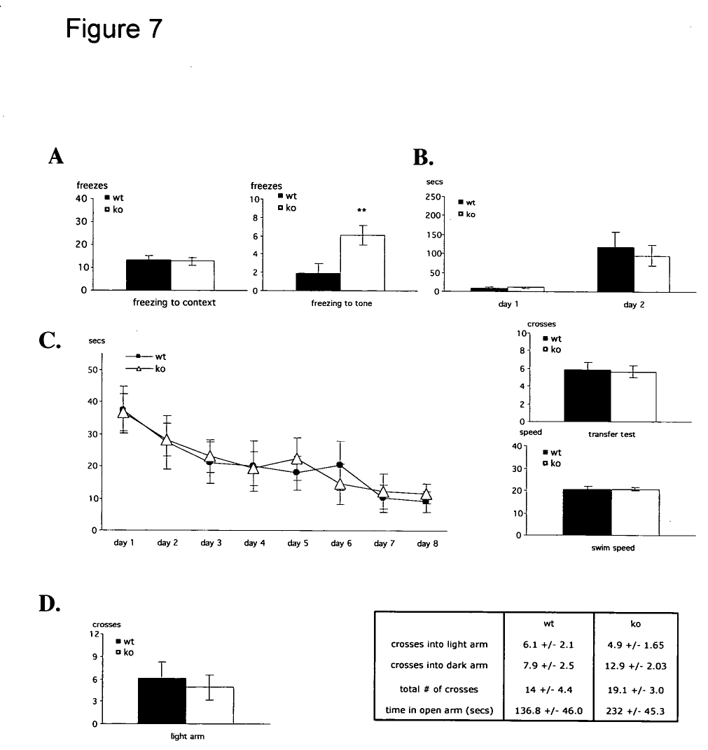

Enhanced associative learning in lynx1 null mutant mice. nAChR activation has been shown to be an important component of specific aspects of learning and memory. Therefore, a series of behavioral tests were run on lynx1 null mutant animals to evaluate learning and memory abilities relative to their wild-type littermates. Mice were trained in a fear-conditioning paradigm, a test of associative and contextual learning. On the training day, an unconditioned stimulus of a mild foot shock was paired with a conditioned stimulus, an innocuous tone. When mice were placed into the identical training environment 24 hr later, lynx1^{-/-} mice and wild-type littermates showed no difference in their freezing response, demonstrating that lynx1^{-/-} mice are normal with respect to contextual learning (FIG. 7A, left). In cue-associated learning, the animals were placed into a novel environment and presented with the shock-associated tone. lynx1^{-/-} mice demonstrated a significant increase in freezing to tone as compared to their wild-type littermates, indicating an alteration in associative learning (FIG. 7A, right). The animals did not show differences when placed in the altered environment, prior to the presentation of tone, indicating no difference in unconditioned fear (data not shown). These data are suggestive of a specific effect of lynx1 on associative fear learning as compared to either unconditioned fear or contextual memory.

To assess the specificity of action of lynx1 in memory processes, lynx1^{-/-} mice were analyzed in two other forms of contextual conditioning: passive avoidance conditioning and Morris water maze learning. In passive avoidance conditioning, mice were placed in the light chamber of a two-chambered box, and the latency to enter into the dark, preferred chamber was measured, whereupon the mice were given a mild foot shock. Lynx1^{-/-} display no differences from wild-type when the latency to enter into the dark chamber was measured the following day (FIG. 7B). Mice were then assessed for performance in the Morris water maze learning task. Mice were trained for 8 days to swim through water to reach a stationary hidden platform, and the latency to find the platform was measured. No significant differences were observed between lynx1^{-/-} and wild-type mice (FIG. 7C), in either the training phase (left), or when the hidden platform was relocated to a different position on the ninth day (the transfer test, upper right). These data are consistent with the lack of effect observed in lynx1 null mutant mice in the contextual component of the fear conditioning task.

These behavioral data are suggestive of a specific involvement of lynx1 in cue-associated learning as opposed to contextual memory. Alternatively, enhanced freezing to tone in lynx1^{-/-} mice could be due to a generalized increase in fear, although the lack of difference in baseline freezing or freezing to context argues against this. To test for differences in anxiety levels, lynx1^{-/-} mice were analyzed in an elevated plus maze paradigm, a more sensitive test for anxiety. Mice were placed for 5 min in a plus-shaped maze which consisted of two open, white arms, and two closed, black arms, and scored for entries into the open arm, entries into the closed arm, and time spent in the open arms. lynx1^{-/-} mice displayed no significant differences from wild-type mice in any of these parameters (FIG. 7D), although lynx1^{-/-} mice displayed a non-significant increase in time spent in the open arm. Therefore lynx1^{-/-} mice manifest no differences in basal levels of anxiety. Thus, increased anxiety is unlikely to account for the freezing to tone observed in the fear conditioning test.

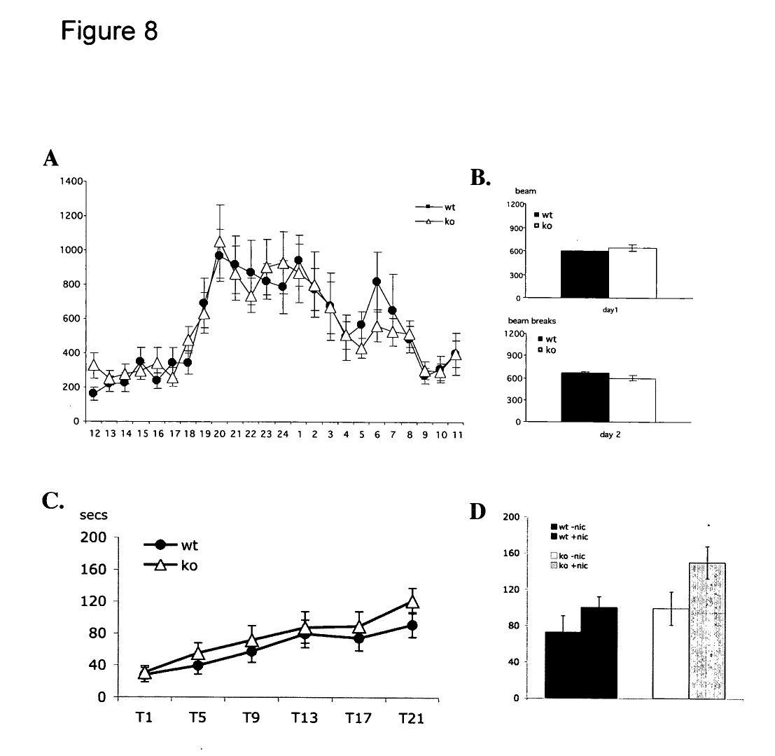

Enhanced behavioral nicotine sensitivity in lynx1 null mutant mice. Nicotine receptor activation has been shown to stimulate locomotor activity in both rats and mice (Clarke and Kumar, 1983, Br. J. Pharmacol. 78, 329-337). To test whether behavioral responses to nicotine were altered in lynx1^{-/-} animals, a series of locomotor tests were performed. To measure general activity levels lynx1^{-/-} mice, they were tested for diurnal locomotor activity over a 72 hr period (FIG. 8A), as well as in a novel environment for 20 min (FIG. 8B). No differences between lynx1⁻ animals and wild-type littermates were observed in either diurnal locomotion (FIG. 6A) or in response to novelty (FIG. 8B), indicating that general activity levels were not significantly altered in lynx1^{-/-} mice.

To test for sensitivity to nicotine, nicotine was administered to lynx1^{-/-} animals and their wild-type littermates chronically (at least 6 weeks). Motor coordination and motor learning were assessed using an accelerating rotarod paradigm. lynx1^{-/-} mice given saccharin alone in their drinking water showed no significant differences in rotarod performance from wild-type mice, either on the initial test day or on subsequent training days (FIG. 8C). Although wild-type mice treated with nicotine plus saccharin (200 μg/ml nicotine in 2% saccharin) showed a trend toward improved performance on the accelerating rotarod compared to saccharin-treated wild-type mice in earlier trials, this difference was not significant (data not shown). In contrast, nicotine-treated lynx1^{-/-} mice displayed a significant improvement in rotarod performance on the 2^{nd} day of training relative to similarly treated wild-type mice, demonstrating a greater effect of nicotine on motor learning in lynx1^{-/-} mice than their wild-type littermates (FIG. 8D). The heightened responsiveness of lynx1^{-/-} mice to nicotine in this motor test is consistent with the observation that cultured neurons from lynx1^{-/-} animals are also more responsive to nicotine (FIG. 5), and with the hypothesis that elimination of lynx1 alters nAChRs toward heightened receptor sensitivity.

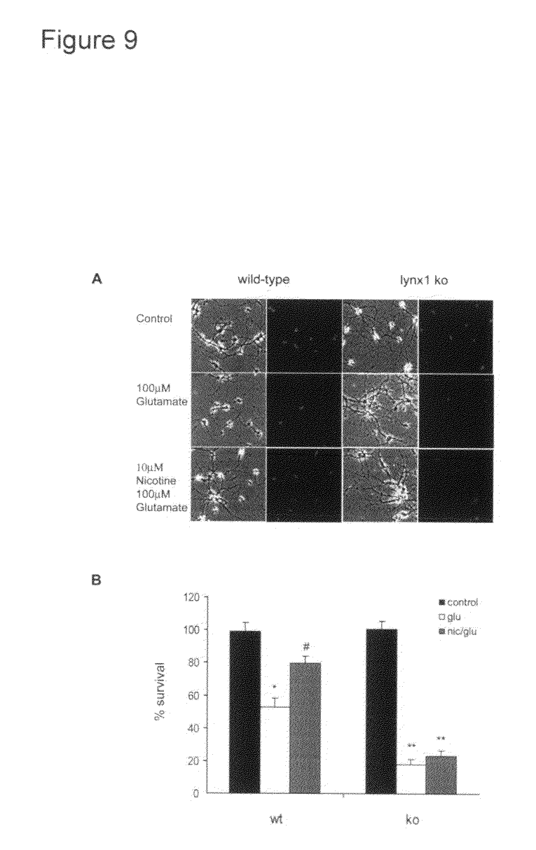

Neurons of lynx1 null mutant mice are more sensitive to excitotoxic insult. Treatment of cultured neurons with glutamate, or glutamate receptor agonists, results in an influx of Ca²⁺ into the cell that can lead to cell death (McLeod et al., 1998, J. Neurophysiol. 80, 2688-2698). Pretreatment of neurons with nicotine prior to glutamate exposure can protect cells from glutamate-mediated excitotoxic cell death (Stevens et al., 2003, J. Neurosci. 23, 10093-10099). Since lynx1^{-/-} cortical neurons showed increased Ca²⁺ accumulation upon nicotine administration, lynx1^{-/-} neurons were more vulnerable to glutamate toxicity, and whether nicotine remains neuroprotective in the absence of lynx1. As shown previously, wild-type cultures exhibited a significant decrease in cell viability upon 100 μM glutamate treatment, and 1 hour pretreatment of nicotine protects wild-type neurons from cell death (FIG. 9A, 9B (left panel)) (Dajas-Bailador et al., 2000, Neuropharmacol. 39, 2799-2807). In contrast, lynx1^{-/-} neurons were more sensitive to glutamate mediated excitotoxicity, and the neuroprotective effect of nicotine was completely abolished (FIG. 9A, B (right panel)). Previous studies have shown that nicotine-mediated neuroprotection usually occurs at low doses of nicotine, and the protective effect of nicotine is eliminated and can even result in more cell death with higher doses of nicotine. Consistent with the idea that the lynx1^{-/-} cultures exhibit a shift in the dose response curve to nicotine, these data suggest that the removal of lynx1 results in heightened sensitivity to nicotine and that a dose of nicotine that is normally neuroprotective is excitotoxic. These data also suggest that there may be an increased vulnerability to neurotoxic insult in lynx1^{-/-} mice, mediated through elevations in Ca²⁺ due to rAChR hyperactivation.

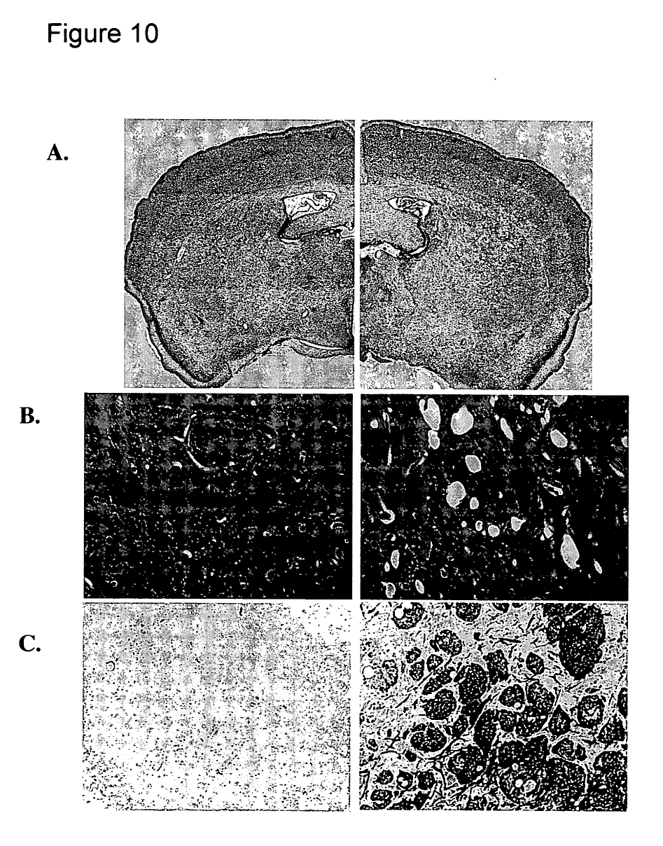

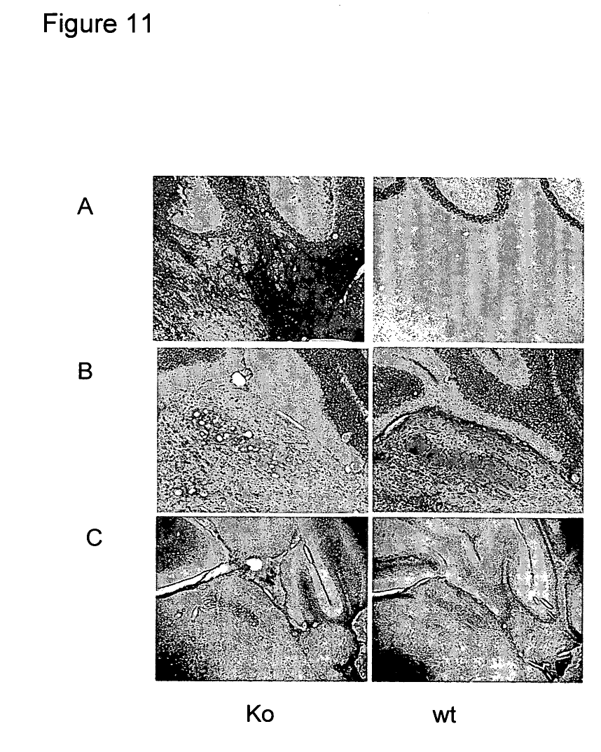

Late onset vacuolating neurodegeneration in lynx1 null mutant mice. Given the enhanced vulnerability of cultured lynx1 null mutant neurons to excitotoxic stimuli, chronic disturbance of nAChR activity evident in lynx1^{-/-} animals might result in cell loss in vivo. Thus, an anatomic study using histological stains on lynx1^{-/-} vs. wild-type coronal brain sections was performed. No significant difference exist between lynx1^{-/-} and wild-type mouse brains at 9 nm ths of age (data not shown). However, inspection of brains from lynx1^{-/-} animals taken at 12 nm ths revealed the presence of large vacuoles, in the dorsal striatum (FIG. 10A), and isolated brainstem regions (data not shown). Most of these lesions were present in the pinker, eosinic areas of the sections, indicating that the degeneration was occurring within axonal tracts within the striatum (FIG. 10B). Further evidence of axonal degeneration was found in the cerebellum (FIG. 11A), where nAChRs have been demonstrated and where vacuolation was found to occur at high levels within the neuropil of the cerebellar lobes (FIG. 11A,B), and the superior cerebellar peduncle (FIG. 11C). To determine whether the vacuolating phenotype of lynx1^{-/-} brains might be affecting neurons, DeOlmos amino cupric silver stain for disintegrative neuronal degeneration were performed on cross sections of 12 month old mutant and wild-type brains. Consistent with the vacuolation within axon dense regions, a predominance of silver staining was observed within axons tracts coursing through the striatum, as well as silver labeling within the corpus callosum (FIG. 10C), and in the cerebellum (FIG. 10D), demonstrating increased neuronal degeneration within aging lynx1 mutant mouse brains.

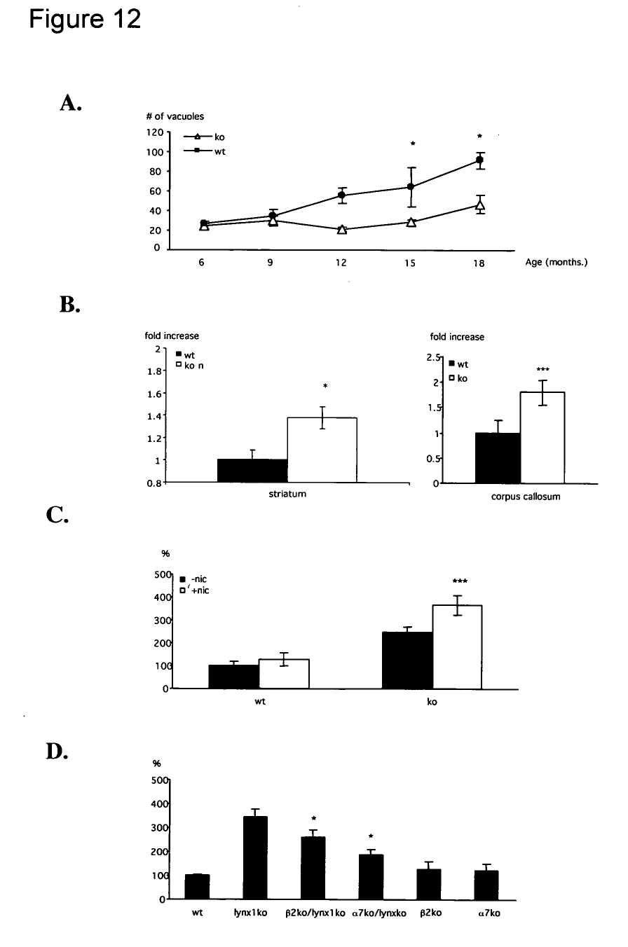

To assess the progressive nature of this degenerative phenotype, the number of lesions present in the striatum of lynx1 mutant mouse brains from 6 to 18 months of age demonstrate an age-dependent increase in vacuolation, first detectable in 12 month old lynx1^{-/-} mice and increasing at 15 and 18 months of age (FIG. 12A). Quantitative analyses of the silver stained sections revealed a significant increase in labeling of lynx1^{-/-} as compared to wild-type brains in the dorsal striatum (FIG. 12B, left) and the medial corpus callosum (FIG. 12B, right). These data are consistent with previous reports that hyper-activation of nAChRs can result in CNS damage, and support the hypothesis that persistent elevations in nicotinic cholinergic signaling make a contribution to neuronal degeneration within aging lynx1^{-/-} mouse brains.

Neurodegeneration in lynx1 null mutant mice is exacerbated by nicotine. If increased activity of nAChRs in lynx1 mutant mice is responsible for this vacuolating phenotype, then pharmacologic manipulations that influence the activity of these receptors might alter the course of degeneration. To test this idea, a solution of nicotine and saccharin, or saccharin alone, was administered to cohorts of lynx1^{-/-} animals and their wild-type littermates through their drinking water. Administration of the nicotine solution was begun at 8 months of age, before the observed onset of degeneration, and continued for a period of 10 months. As shown in FIG. 12C, there were no significant differences in vacuolation in the striatum of saccharin vs. nicotine/saccharin-treated wild-type mice (left). However, in lynx1^{-/-} mice treated with the nicotine solution, a significant increase in vacuolation (FIG. 12C, right) was observed. These data strongly suggest that the degenerative phenotype observed in lynx1^{-/-} brains results from increased nAChR activity, consistent with the enhanced vulnerability of lynx1^{-/-} neurons to excitotoxic stimuli and the loss of neuroprotective effect documented above (FIG. 7).

Neurodegeneration in lynx1 null mutant mice requires nAChRs. Previous in vitro studies, including single channel recordings of nAChR activity with and without lynx 1, demonstrated a direct effect of lynx1 on nAChRs. If the degenerative phenotype of lynx1^{-/-} mice reflects the loss of its ability to modulate nAChR activity, then deletion of nAChRs would be expected to rescue the degenerative phenotype observed in lynx1^{-/-} animals. To test this idea, lynx1 null mutations were crossed onto nAChR mutant backgrounds to prepare double mutant animals in which the effect of nAChRs on the lynx1^{-/-} degenerative phenotype could be assessed. As shown in FIG. 12D, a significant reduction in the number of lesions present in the striatum at 15 months of age was observed in both lynx1/β2 nAChR^{-/--/-} and lynx1/α7 nAChR^{-/--/-} double mutant mice relative to their littermates bearing only the lynx1^{-/-} mutation. It is notable that the rescue of degeneration in lynx1/α7 nAChR^{-/--/-} was greater than that observed lynx1/β2 nAChR^{-/--/-} double mutant animals, since a7 nAChRs have a greater Ca²⁺ conductance than β2 nAChRs (Berg and Conroy, 2002, J. Neurobiol. 53, 512-523). These data demonstrate that lynx1 action in vivo requires α7 and β2 nAChRs. Taken together with the in vitro data presented previously and in the current study, and the differential effects of nicotine treatment on motor learning and neurodegeneration observed in lynx1^{-/-} animals (FIGS. 6 and 10), the rescue of the degenerative phenotype in lynx1 mutant animals by deletion of nAChRs provides a strong argument for a direct role of lynx1 in modulation of nAChR activity in vivo.

The analyses of lynx1 null mutant mice reveal several important new features of lynx1 function and its impact on cholinergic activity in the central nervous system. Whole-cell recordings of responses to nicotine pulses in brain slices show that loss of lynx1 results in hypersensitivity of nAChRs to nicotine, and to prolonged nAChR receptor activation. These changes are sufficient to raise intracellular Ca²⁺ levels in lynx1 null mutant but not in wild-type neurons, in response to acute or maintained nicotine. lynx1 null mutant mice exhibit a reduction in paired-pulse facilitation ratios in brain slices, indicating increased synaptic efficacy within neuronal ensembles. lynx1 mutant mice perform better than wild-type littermates on specific tasks of associative learning, and lynx1 mutant mice are more responsive to nicotine in a motor learning paradigm. Finally, loss of lynx1 modulation leads to increased vulnerability to excitotoxic stimuli and loss of the neuroprotective effect of nicotine. Accordingly, aging lynx1 null mutant mice suffer from a progressive, vacuolating degeneration of the brain which is exacerbated by nicotine administration and rescued by null mutations in nAChRs.

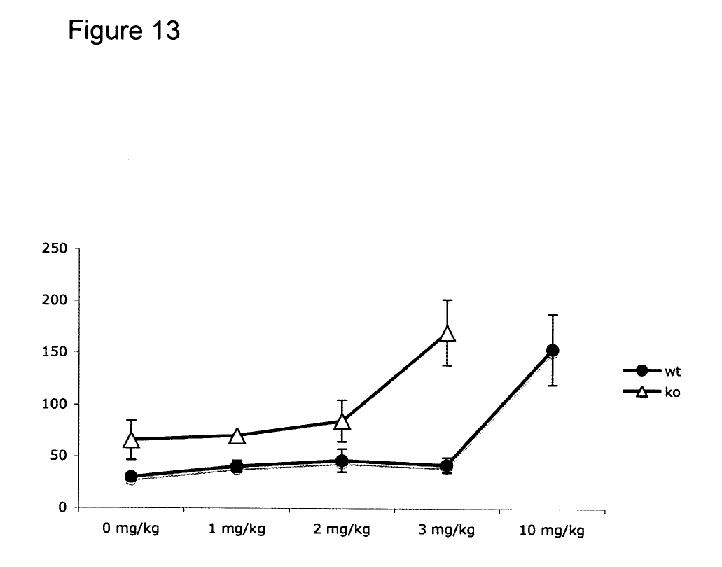

Nicotine receptor activation has been shown to have analgesic properties, and null mutant mice in the lynx1 gene display abnormal antinociception. FIG. 13 shows that lynx1 null mutant mice earlier onset of nicotine-mediated antinociception. Time on a hot-plate was used to assess pain sensitivity and the antinociceptive effect of nicotine (in mg/kg in PB). lynx1 null mutant mice demonstrate an enhanced sensitivity to the antinociceptive properties of nicotine.

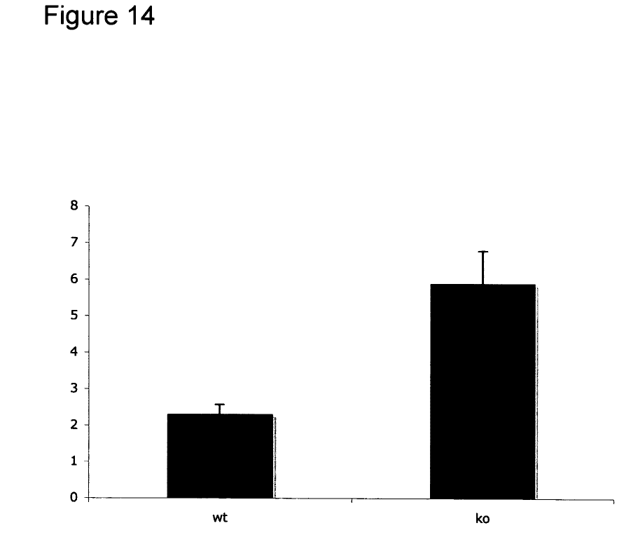

FIG. 14 shows that lynx1 null mutant mice are more sensitive to nicotine induced seizures that wt mice. Seizure index is on a scale of 1-8, and indicated the extent of pre-seizure or seizure activity in response to a single injection of nicotine (mg/kg weight).

### REFERENCES CITED HEREIN

Rezvani and Levin, 2001, Biol Psych. 49, 258-267,
Picciotto et al., 2000, Neuropsychopharmacol. 22, 451-465
US Department of Health and Human Services, 1988, U.S. Government Printing Office.
Lindstrom, 1997, Mol. Neurobiol. 15, 193-222
Damaj et al., 1999, J. Pharmacol. Exp. Ther. 291, 1284-1291
Abrous et al., 2002, J. Neurosci. 22, 3656-3662
Fonck et al., 2003, J. Neurosci. 23, 2582-2590
Fonck et al., 2005, J. Neurosci. 25, 11396-11411,
Broide et al., 2002, Mol. Pharmacol. 61, 695-705
Orr-Urtreger et al., 2000, J. Neurochem. 74, 2154-2166
Dani et al., 2000, Eur. J. Pharmacol. 393, 31-38,
Wooltorton et al., 2003, J. Neurosci. 23, 3176-3185
Kuryatov, et al., 1997, J. Neurosci. 17, 9035-9047
Miwa et al., 1999, Neuron 23, 105-114
Ibanez-Tallon et al., 2002, Neuron 33, 893-903
Ibanez-Tallon et al, 2004, Neuron 43, 305-311
Dessaud et al, 2006, Mol. Cell. Neurosci. 2006, 31, 232-242
Gumley et al., 1995, Immunognetics 42, 221-224
Fleming et al., 1993, J. Immunol. 150, 5379-5390
Ploug and Ellis, 1994, FEBS Lett. 349, 163-168
Adermann et al., 1999, Protein Sci. 8, 810-819
Arredondo et al., 2006, J. Cell Physiol. 208, 238-45
Kawashima et al., 2007, Life Sci., 80, 2314-2319
Rees et al., 1987, Proc. Natl. Acad. Sci. 84, 3132-3136
Love and Stroud, 1986, Pro. Engineer. 1, 37-46
Fletcher et al., 1994, Structure, 2, 185-199
Miwa et al., 2006, Neuron, 51, 587-600
Sandberg et al., 1998, J. Med. Chem. 41. 2481-2491
Sambrook et al., 1985, Glover (ed.). MRL Press, Ltd., Oxford, U.K., vol. I, II
Weis et al., 1992, Trends Genet, 8, 263-264
Frohman, 1994, PCR Methods Appl., 4, S40-58.
Cale et al., 1998, Methods Mol. Biol., 105, 351-71
Frohman (1994), PCR Methods Appl., 4, S40-58
Benton and Davis. (1977), Science, 196, 180-182
Clemmons (1993), Mol. Reprod. Dev., 35, 368-374
Loddick et al., (1998), Proc. Natl. Acad. Sci., U.S.A., 95, 1894-1898
Protein Purification Protocols (Methods in Molecular Biology), Cutler, P., (ed.), 2003, Humana press.
Cale et al., 1998, Methods Mol. Biol., 105, 351-371
Hopp and Woods, 1981, Proc. Natl. Acad. Sci., U.S.A., 78, 3824
Chou and Fasman, 1974, Biochem., 13, 222-245
Swift et al., 1984, Cell, 38, 639-646
Hanahan, 1985, Nature, 315, 115-122
Grosschedl et al., 1984, Cell, 38, 647-658
Leder et al. 1986, Cell, 45, 485-495
Pinkert et al., 1987, Genes Dev., 1, 268-276
Krumlauf et al., 1985, Mol. Cell. Biol., 5, 1639-1648
Kelsey et al., 1987, Genes Dev., 1, 161-171
Magram et al., 1985, Nature, 315, 338-340
Readhead et al., 1987, Cell, 48, 703-712
Shani, 1985, Nature, 314, 283-286
Mason et al., 1986, Science, 234, 1372-1378
Smith and Johnson, 1988, Gene, 67, 31-40
Lei et al., 1987, J. Bacteriol., 169, 4379
Kim et al., 1997, Mol. Immunol., 34, 891
Chan et al, 2001, Cytometry, 44, 361-368
Hu et al., 2007, Proteomics, 7, 2151-2161
Lin et al., 2005, Toxicon, 47, 265-270
Sekine-Aizawa and Huganir, 2004, Proc. Natl. Acad. Sci., 101, 17114-17119
Stiles, 1993, Toxicon, 31, 825-834
Chung, et al., 2007, Biophys. J., 93, 1747-1757,
Salminen et al., 2005, Neuropharmacol., 48, 696-705
Hille, B., 1992, Ion Channels of Excitable Membranes, Sinauer Associates
Hamill, et al, 1981, Pflugers Arch., 391, 85-100,
Li et al., 2006, Nano Lett. 6, 815-819
Kandel et al., 2000, Principles of Neural Science, 4th ed., McGraw-Hill, New York,
Modern Techniques in Neuroscience Research, Windhorst and Johansson, (Eds), 1999, Springer Lab Manuals, 146-155
Modern Techniques in Neuroscience Research, Windhorst, and Johansson, (Eds), 1999,
Springer Lab Manuals, 311-318)
Modern Techniques in Neuroscience Research, Windhorst and Johansson, (Eds), 1999, Springer Lab Manuals, 134-146
Steidl et al., 2006, Brain Res., 1096, 70-84
Ding et al., 2007, Neurosci Lett. 422, 175-180
Nashmi et al. 2003, J. Neurosci. 23, 11554-11567
Gill et al., 2007, Assay Drug Dev. Technol., 5, 373-380
Vijayaraghavan et al., 1992, Neuron, 8, 353-362
Single Channel Recording, Sakmann and Neher, (eds.), 1983, 135-174, Plenum, New York
Wang et al., 2007, Neurosci Lett., 2, 133-138
Crawley, 2007, What's Wrong With My Mouse? Behavioral Phenotyping of Transgenic and Knockout Mice. Second Edition. John Wiley & Sons, Hoboken N.J.
Langer, supra; Sefton, 1987, CRC Crit. Ref. Biomed. Eng. 14, 201
Buchwald et al., 1980, Surgery 88, 507
Saudek et al., 1989, N. Engl. J. Med. 321, 574
Medical Applications of Controlled Release, Langer and Wise (eds.), 1974. CRC Pres., Boca Raton, Fla.;
Controlled Drug Bioavailability, 1984. Drug Product Design and Performance, Smolen and Ball (eds.), Wiley, New York
Ranger and Peppas, 1983, Macromol. Sci. Rev. Macromol. Chem. 23, 61
Levy et al., 1985, Science, 228, 190
During et al., 1989, Ann. Neurol. 25, 351
Howard et al., 1989, J. Neurosurg. 71, 105
Goodson, 1984. in Medical Applications of Controlled Release, vol. 2. 115-138
Langer, 1990. Science, 249, 527-1533
Remington's Pharmaceutical Sciences by E. W. Martin
Clarke and Kumar, 1983, Br. J. Pharmaol. 78, 329-337
Dajas-Bailador et al., 2000, Neuropharmacol. 39, 2799-2807
Berg and Conroy, 2002, J. Neurobiol. 53, 512-523
U.S. Pat. No. 6,514,947
U.S. Pat. No. 5,770,580

All references, including patents, articles, books, and chapters cited herein are hereby incorporated in their entirety by reference herein.

What is claimed:

1. A method for treating a neurological disorder in a subject, the method comprising administering an effective amount of a lynx1, lynx2, or lynx 3 polypeptide or nucleic acid compositions to a subject suffering from said neurological disorder.

2. The method of claim 1, wherein said lynx composition is a lynx1 polypeptide or nucleic acid.

3. The method of claim 1, wherein said lynx composition is a lynx2 polypeptide or nucleic acid or a lynx3 polypeptide or nucleic acid.

4. The method of claim 1, wherein the lynx1, lynx2, or lynx 3 composition is a polypeptide or nucleic acid composition that modulates the specific activity of nicotinic acetylcholine receptors or the specific activity of functionally or structurally related proteins.

5. The method of claim 1, wherein the method cures, lessens the severity, shortens the duration, delays or prevents the onset, or ameliorates the symptoms of said neurological disorder.

6. The method of claim 1, wherein the neurological disorder is the result of endogenous nicotinic acetylcholine receptor dysregulation or dysfunction.

7. The method of claim 1, wherein the neurological disorder is not the result of endogenous nicotinic acetylcholine receptor dysregulation or dysfunction.

8. The method of claim 1, wherein the lynx1, lynx2, or lynx 3 is a polypeptide composition having activity in neurological processes selected from the group consisting of cognition, learning or memory.

9. The method of claim 1, wherein the neurological disorder is a mood or anxiety disorder.

10. The method of claim 9, wherein the neurological disorder is selected from the group consisting of depression, anxiety, attention deficit hyperactivity disorder, attention deficit disorder, post-traumatic stress disorder, Tourette's, delirium, pain, bipolar disorder, and mania.

11. The method of claim 1, wherein the neurological disorder is a neurodegenerative disorder.

12. The method of claim 11, wherein the neurological disorder is selected from the group consisting of age associated memory impairment, mild cognitive impairment, Parkinson's disease, Alzheimer's disease, progressive supranuclear palsy, dementia with Lewy Bodies, stroke, and Huntington's disease.

13. The method of claim 1, wherein the neurological disorder is selected from the group consisting of dyslexia, autism, schizophrenia, epilepsy, neuropathic pain, smoking cessation, addiction, and alcoholism.

14. The method of claim 1, wherein the lynx1, lynx2, or lyxn3 composition is a mature lynx polypeptide or a polynucleotide that causes the expression of a mature lynx polypeptide.

15. The method of claim 1, wherein the composition is administered to the central nervous system of the subject or delivered in a way that it is active only in the central nervous system.

16. The method of claim 1, wherein the method comprises administering a nucleic acid expression vector capable of expressing a lynx polypeptide in the CNS of the subject.

17. The method of claim 1, claim 2, or claim 3, wherein the subject is a mammal.

18. The method of claim 17, wherein the mammal is a human.


SEQUENCE LISTING (19 sequences)

SEQ ID NO: 1 - protein, 131 aa, Homo sapiens
MTPLLTLILVVLMGLPLAQALDCHVCAYNGDNCFNPMRCPAMVAYCMTTRTSAAEAIWCHQCTGFGGCSHGSRCLRDSTHCVTTATRVLSNTEDLPLVTKMCHIGCPDIPSLGLGPYVSIACCQTSLCNHD

SEQ ID NO: 2 - protein, 116 aa, Homo sapiens
MTPLLTLILVVLMGLPLAQALDCHVCAYNGDNCFNPMRCPAMVAYCMTTRTYYTPTRMKVSKSCVPRCFETVYDGYSKHASTTSCCQYDLCNGTGLATPATLALAPILLATLWGLL

SEQ ID NO: 3 - protein, 116 aa, Mus musculus
MTHLLTVFLVALMGLPVAQALECHVCAYNGDNCFKPMRCPAMATYCMTTRTYFTPYRMKVRKSCVPSCFETVYDGYSKHASATSCCQYYLCNGAGFATPVTLALVPALLATFWSLL

SEQ ID NO: 4 - protein, 141 aa, Homo sapiens
MWVLGIAATFCGLFLLPGFALQIQCYQCEEFQLNNDCSSPEFIVNCTVNVQDMCQKEVMEQSAGIMYRKSCASSAACLIASAGYQSFCSPGKLNSVCISCCNTPLCNGPRPKKRGSSASALRQGLRTTILFLKLALFSAHC

SEQ ID NO: 5 - protein, 141 aa, Mus musculus
MWVLGIAATFCGLFWLPGLALQIQCYQCEEFQLNNDCSSPEFIVNCTVNVQDMCQKEVMEQSAGIMYRKSCASSAACLIASAGYQSFCSPGKLNSVCISCCNTPLCNGPRPKKRGSSASAIRPGLLTTLLFFHLALCLAHC

SEQ ID NO: 6 - protein, 125 aa, Homo sapiens [sequence omitted]

SEQ ID NO: 7 - protein, 127 aa, Mus musculus
MQGTWMVLLALILGTFGELAMALQCYTCANPVSASNCVTTTHCHINETMCKTTLYSLEIVFPFLGDSTVTKSCASKCEPSDVDGIGQTRPVSCCNSDLCNVDGAPSLGSPGGLLLALALFLLLGVLL

SEQ ID NO: 8 - DNA, 1352 bp, Homo sapiens
ttagggtagaagaaaaggttttatttttctttctcacattggaaaaaatgaaaactttcggacccatgaaattttattacattttgccaaaaacagaaccaataacataagtattcaaagttatgtaaagataattatttaatatgaacattatgatggtgagagggaccacggagcaaggggctgccttgcaggcctgccttccagctttgctacagggaccagaagcgggagctgagcgcaggggaggcaggcggaggccatgggcagcgaggcgggtgcgccaaagggcgccagttccggagctgctgggcctgggctgcaggagggcggagctggggccgcaaggctggtgccggccgacaaacgacgcggcgcgggcgagtgggcggaggcggctgcggggaaggctgggctgccgcgggacgcggtgaagatagcctgcggagtgtccgggctgaacacggttgcagcactcccagtagaccaggagctccgggaggcagggccggccccacgtcctctgcgcaccaccctgagttggatcctctgtgcgccacctgagttggatccagggctagctgctgttgacctccccactcccacgctgccctcctgcctgcagccatgacgcccctgctcaccctgatcctggtggtcctcatgggcttacctctggcccaggccttggactgccacgtgtgtgcctacaacggagacaactgcttcaaccccatgcgctgcccggctatggttgcctactgcatgaccacgcgcacctctgcagccgaagccatatggtgtcaccagtgcacgggcttcggagggtgctcccatggatccagatgcctgagggactccacccactgtgtcaccactgccacccgggtcctcagcaacaccgaggatttgcctctggtcaccaagatgtgccacataggctgccccgatatccccagcctgggcctgggcccctacgtatccatcgcttgctgccagaccagcctctgcaaccatgactgacggctgccctcctccaggcccccggacgctcagcccccacagcccccacagcctggcgccagggctcacagctgcccctccctcgagactggccagcccacctctcccggcctctgcagccaccgtccagcaccgcttgtcctagggaagtcctgcgtggagtcttgcctcaatctgctgccgtccaagcctggggcccatcgtgcctgccgccccttcaggtcccgacctccccacaataaaatgtgattggatcgtgtggtacaaaaaaaaaaaaaaaaaaaaaaaaaaaaaaaaaaaaaaaaaaaaaaaaaaaaaaaaaaaaaaaa

SEQ ID NO: 9 - DNA, 979 bp, Homo sapiens
cgcgggcgagtgggcggaggcggctgcggggaaggctgggctgccgcgggacgcggtgaagatagcctgcggagtgtccgggctgaacacggttgcagcactcccagtagaccaggagctccgggaggcagggccggccccacgtcctctgcgcaccaccctgagttggatcctctgtgcgccacccctgagttggatccagggctagctgctgttgacctccccactcccacgctgccctcctgcctgcagccatgacgcccctgctcaccctgatcctggtggtcctcatgggcttacctctggcccaggccttggactgccacgtgtgtgcctacaacggagacaactgcttcaaccccatgcgctgcccggctatggttgcctactgcatgaccacgcgcacctctgcagccgaagccatatggtgtcaccagtgcacgggcttcggagggtgctcccatggatccagatgcctgagggactccacccactgtgtcaccactgccacccgggtcctcagcaacaccgaggatttgcctctggtcaccaagatgtgccacataggctgccccgatatccccagcctgggcctgggcccctacgtatccatcgcttgctgccagaccagcctctgcaaccatgactgacggctgccctcctccaggcccccggacgctcagcccccacggcccccacagcctggcgccagggctcacggccgcccctccctcgagactggccagcccacctctcccggcctctgcagccaccgtccagcaccgcttgtcctagggaagtcctgcgtggagtcttgcctcaatctgctgccgtccaagcctggggcccatcgtgcctgccgccccttcaggtcccgacctccccacaataaaatgtgattggatcgtgtggtacaaaaaaaaaaaaaaaaaaaaaaaaaaaaaaaaaaaaaaaaaaaaaaaaaaaaaaaaaaaaaaaa

SEQ ID NO: 10 - DNA, 4967 bp, Homo sapiens
tttagggtagaagaaaaggttttatttttctttctcacattggaaaaaatgaaaactttcggacccatgaaattttattacattttgccaaaaacagaaccaataacataagtattcaaagttatgtaaagataattatttaatatgaacattatgatggtgagagggaccacggagcaaggggctgccttgcaggcctgccttccagctttgctacagggaccagaagcgggagctgagcgcaggggaggcaggcggaggccatgggcagcgaggcgggtgcgccaaagggcgccagttccggagctgctgggcctgggctgcaggagggcggagctggggccgcaaggctggtgccggccgacaaacgacgcggcgcgggcgagtgggcggaggcggctgcggggaaggctgggctgccgcgggacgcggtgaagatagcctgcggagtgtccgggctgaacacggttgcagcactcccagtagaccaggagctccgggaggcagggccggccccacgtcctctgcgcaccaccctgagttggatcctctgtgcgccacctgagttggatccagggctagctgctgttgacctccccactcccacgctgccctcctgcctgcagccatgacgcccctgctcaccctgatcctggtggtcctcatgggcttacctctggcccaggccttggactgccacgtgtgtgcctacaacggagacaactgcttcaaccccatgcgctgcccggctatggttgcctactgcatgaccacgcgcacctactacacccccaccaggatgaaggtcagtaagtcctgcgtgccccgctgcttcgagactgtgtatgatggctactccaagcacgcgtccaccacctcctgctgccagtacgacctctgcaacggcaccggccttgccaccccggccaccctggccctggcccccatcctcctggccaccctctggggtctcctctaaagcccccgaggcagacccactcaagaacaaagctctcgagacacactgctacaccctcgcacccagctcaccctgcctcaccctccacactccctgcgacctcctcagccatgcccagggtcaggactgtgggcaagaagacacccgacctcccccaaccaccacacgacctcacttcgaggccttgacctttcgatgctgtgtgggatcccaaaagtgtccggctttgatgggctgatcagcccctcgcctgtccagggctatttatggggaggggcccagtcaggcagggagcactgagcagatgggagctgtggccaagcccaccttctgggaagccctaggagaggcccctgcctcagtctgcccctggtgtgctggggcagggggaagacaaggaatgcccgcaggtggggtggtggggagactcttaggaggaaaggctcctccaggcctagtcacgcttcctactgaggccaggagctgccaggaccggtacgggatcagggctgtgggaggagggcctttggcaccggcccctgtgctaggaagtctgccaggcccgagttggagccaccccctgcaggggagggggcggctctgcctcagcaggccccagggcccccgaagtcacagaagctttttcgggtccagcaaggggtgtgtgtcctctcagtcaaaccccttgacgtttcccaccccctcacggggagggcaccaggcctgaagctggcaggagctagggccatgctatatttggtgggtcctggacgctgacccggccagcgctattctgggcagggagggaaaggggcagagcaggtggtcccccgagtcctggtccccaaccacagcaggacccagccgagcaaggcaaaagacgcaggactgggggatgcgcgcacaggctgggggttgggagcagcctggggccggcgcgggcctgggcgtgggaaggcggagcatgccaccctctcgtggccgtgcgggggcgggagtggggagggtggggtggggagggcggagacccagcccctcccccgcagcgggatgcgcacagtgagtgggtccctccatcaccttccacctggggatgcacccactgggagggcagggtggaagccccagctgggtgtgtggactcccagggaccccaccccaggcctgggaagcaggggtcagcccaacacgcaccaatccatccccgatgcaggtagcccagggagcacctgcccctgctgtgaatggggcattcgggggcgtgagaccttttggtgccagcggtcacgctgcacctgaggcccccacctgaccagtgctcccagctctggtgtcctgagaaacccttcaagccatcccgcatgggcaggatggtgacatatccatgtggtaggattgtcccggccccaaagtatggccctggtcaggggagcccctgctggaaattgcatctccagagctttgatgcaggacccctgggggatcagggaatgagggtctccaccccaggggtctccttgcagtgagtctatatgcaggcctgcgttctgctcctggggctggttctgagtgcccagcttcagtctcctgagaacatgaggatgggagggggcagagtcttgctgagggcacacccagttcccgctggaggaggacagtgccagtcttctgcaaagggaccttgggtgggaacgggcccggagcgggaggaacgtgactccccagagggaagatgggcatcatactgggcccagagctgggaaggagttgctgccagcacagggtgggcctggactcccctcgcccctacccccagtggttgtggctgtagccctaagcctggagagcaggaccggcccggggtgtctgggaggctgccaggtgcctcccagagctcccaagggcccccacctgcaagtgccagcctcagggcagtgcccaaatgaggccctctcagctgcagccagcgatgccttgggatgcttaccgggagggaggcggctttgggctcctaagtccttgggagaggctgggagcagtcactgcgcggcttgcgcaagcccattgtcgggttgggtggcttcctcagccagggctgggagggactccaggatcaggtcctccctgtctcgagtctcagtggggtgatggggaggagacctggccacccatggctcaggggcagctgagaacaaggacctgctggagctggaagtgctgtggtgttgaggggtggggtgggcagcttctcacacctgcctcctgcctccttctgtccacctttccaccaccctgacctgtcccagccccacacatggttctgcctggctggcctgcccttggcacctggcgtagagcacacagaaggcactcagctaatgctgggcaggcccactcatggggagtgcgtggctgtgcagcaccagggaaccggcacagcagcgccggcagaaatcacagcagtaaacttgtccgggttgtatgcatcaaggtggcgatggacgtgggtccccccactgcactgtggccctgagcactgtatagcagcccggcaatgggagccattatcttgcccctttgacagaggaggacacagaggcacagggaggtgaagtagctgccccacactagtgcctcctcgctcactcaccaccccctgcaccacagtgcagccgcttctcccaccagctggggttccttggacccccaagcctgggaagggggaggtgagtttacaaaatggaaagcttaaaaggagaaaagtggaaccagaggtttgagaagccctgagtggtagagtaaggcctccagcgctgcctctgggtgcagggcagagtggcagaggagagggggagaggcactgggcaccatgggggcccagttcccacttcggggatctctctcgcagaaccgagggtccccttcatgggggtagatgcccagggctagctgttgccactgtctgtgtggacctgagtcctggacatgcccgagtgactcaggagtggctgcttgggcgggctctgtcaccctaggatgttatacattctgggaactggacaggagtggctgcttgggcgggctctggcaccctgggatgttatacattctgggaactggacaggagtggctgcttgggtgggctctggcaccctgggatgttatacattctgggaactgcaatcagccactagagaagtcggagctacaggaagtgaccctggggtgggacctggggacatggccaggtcagcatggggacacccggctccagcaggagctctggtctgtcctggggtctttgggggcagggctgcggccctgggcaggcttcctccaggcggaggtcctggggaagtgggggagccaggccagctgccgcctcccccactatgtagcatctgattcgtcatctctcatgaaggcgatttggttcataactctgaaactctgaaaaaggtcaaaagaagcagagaggccctcggtggatatgccagcttttctgccggtgctttctcccactactctgggtggtctgctctcctcttcaaacctcagctcgcagggagggcctgaatctgccagcccctcaggatctccttccctctgggccctccccagccttaaggagcctcccagacagaagggtggacagagccacctgggcagcccgagagacacacgggggtcctccctgtggacagccctgccagcttccgcccagccctgagcttcatttgcatcttgaggagtaaggggtggtgaaatgggaatgctggtctggctcagctggtcgtgggcataagtgcccgctgaatggatggcatctctccctcctgtcttatgttctggggtccaggtgcttcccagggccatgcccctgctgctaatgcttgccctaacccttaccctaaccagcgtccagcgtcgtctcaccgagccgtaaataaatcaacagattcgcattgtcaaaaaaaaaaaaaaaaaa

SEQ ID NO: 11 - DNA, 4609 bp, Homo sapiens
agggggttgggaagctcgaagccgcaggcctgactctggcctttggcatcctggggttggcctgggcaaatgtgtcgtgagagacggatttgttgttctcgggaaggcggttgcagcactcccagtagaccaggagctccgggaggcagggccggccccacgtcctctgcgcaccaccctgagttggatcctctgtgcgccacctgagttggatccagggctagctgctgttgacctccccactcccacgctgccctcctgcctgcagccatgacgcccctgctcaccctgatcctggtggtcctcatgggcttacctctggcccaggccttggactgccacgtgtgtgcctacaacggagacaactgcttcaaccccatgcgctgcccggctatggttgcctactgcatgaccacgcgcacctactacacccccaccaggatgaaggtcagtaagtcctgcgtgccccgctgcttcgagactgtgtatgatggctactccaagcacgcgtccaccacctcctgctgccagtacgacctctgcaacggcaccggccttgccaccccggccaccctggccctggcccccatcctcctggccaccctctggggtctcctctaaagcccccgaggcagacccactcaagaacaaagctctcgagacacactgctacaccctcgcacccagctcaccctgcctcaccctccacactccctgcgacctcctcagccatgcccagggtcaggactgtgggcaagaagacacccgacctcccccaaccaccacacgacctcacttcgaggccttgacctttcgatgctgtgtgggatcccaaaagtgtccggctttgatgggctgatcagcccctcgcctgtccagggctatttatggggaggggcccagtcaggcagggagcactgagcagatgggagctgtggccaagcccaccttctgggaagccctaggagaggcccctgcctcagtctgcccctggtgtgctggggcagggggaagacaaggaatgcccgcaggtggggtggtggggagactcttaggaggaaaggctcctccaggcctagtcacgcttcctactgaggccaggagctgccaggaccggtacgggatcagggctgtgggaggagggcctttggcaccggcccctgtgctaggaagtctgccaggcccgagttggagccaccccctgcaggggagggggcggctctgcctcagcaggccccagggcccccgaagtcacagaagctttttcgggtccagcaaggggtgtgtgtcctctcagtcaaaccccttgacgtttcccaccccctcacggggagggcaccaggcctgaagctggcaggagctagggccatgctatatttggtgggtcctggacgctgacccggccagcgctattctgggcagggagggaaaggggcagagcaggtggtcccccgagtcctggtccccaaccacagcaggacccagccgagcaaggcaaaagacgcaggactgggggatgcgcgcacaggctgggggttgggagcagcctggggccggcgcgggcctgggcgtgggaaggcggagcatgccaccctctcgtggccgtgcgggggcgggagtggggagggtggggtggggagggcggagacccagcccctcccccgcagcgggatgcgcacagtgagtgggtccctccatcaccttccacctggggatgcacccactgggagggcagggtggaagccccagctgggtgtgtggactcccagggaccccaccccaggcctgggaagcaggggtcagcccaacacgcaccaatccatccccgatgcaggtagcccagggagcacctgcccctgctgtgaatggggcattcgggggcgtgagaccttttggtgccagcggtcacgctgcacctgaggcccccacctgaccagtgctcccagctctggtgtcctgagaaacccttcaagccatcccgcatgggcaggatggtgacatatccatgtggtaggattgtcccggccccaaagtatggccctggtcaggggagcccctgctggaaattgcatctccagagctttgatgcaggacccctgggggatcagggaatgagggtctccaccccaggggtctccttgcagtgagtctatatgcaggcctgcgttctgctcctggggctggttctgagtgcccagcttcagtctcctgagaacatgaggatgggagggggcagagtcttgctgagggcacacccagttcccgctggaggaggacagtgccagtcttctgcaaagggaccttgggtgggaacgggcccggagcgggaggaacgtgactccccagagggaagatgggcatcatactgggcccagagctgggaaggagttgctgccagcacagggtgggcctggactcccctcgcccctacccccagtggttgtggctgtagccctaagcctggagagcaggaccggcccggggtgtctgggaggctgccaggtgcctcccagagctcccaagggcccccacctgcaagtgccagcctcagggcagtgcccaaatgaggccctctcagctgcagccagcgatgccttgggatgcttaccgggagggaggcggctttgggctcctaagtccttgggagaggctgggagcagtcactgcgcggcttgcgcaagcccattgtcgggttgggtggcttcctcagccagggctgggagggactccaggatcaggtcctccctgtctcgagtctcagtggggtgatggggaggagacctggccacccatggctcaggggcagctgagaacaaggacctgctggagctggaagtgctgtggtgttgaggggtggggtgggcagcttctcacacctgcctcctgcctccttctgtccacctttccaccaccctgacctgtcccagccccacacatggttctgcctggctggcctgcccttggcacctggcgtagagcacacagaaggcactcagctaatgctgggcaggcccactcatggggagtgcgtggctgtgcagcaccagggaaccggcacagcagcgccggcagaaatcacagcagtaaacttgtccgggttgtatgcatcaaggtggcgatggacgtgggtccccccactgcactgtggccctgagcactgtatagcagcccggcaatgggagccattatcttgcccctttgacagaggaggacacagaggcacagggaggtgaagtagctgccccacactagtgcctcctcgctcactcaccaccccctgcaccacagtgcagccgcttctcccaccagctggggttccttggacccccaagcctgggaagggggaggtgagtttacaaaatggaaagcttaaaaggagaaaagtggaaccagaggtttgagaagccctgagtggtagagtaaggcctccagcgctgcctctgggtgcagggcagagtggcagaggagagggggagaggcactgggcaccatgggggcccagttcccacttcggggatctctctcgcagaaccgagggtccccttcatgggggtagatgcccagggctagctgttgccactgtctgtgtggacctgagtcctggacatgcccgagtgactcaggagtggctgcttgggcgggctctgtcaccctaggatgttatacattctgggaactggacaggagtggctgcttgggcgggctctggcaccctgggatgttatacattctgggaactggacaggagtggctgcttgggtgggctctggcaccctgggatgttatacattctgggaactgcaatcagccactagagaagtcggagctacaggaagtgaccctggggtgggacctggggacatggccaggtcagcatggggacacccggctccagcaggagctctggtctgtcctggggtctttgggggcagggctgcggccctgggcaggcttcctccaggcggaggtcctggggaagtgggggagccaggccagctgccgcctcccccactatgtagcatctgattcgtcatctctcatgaaggcgatttggttcataactctgaaactctgaaaaaggtcaaaagaagcagagaggccctcggtggatatgccagcttttctgccggtgctttctcccactactctgggtggtctgctctcctcttcaaacctcagctcgcagggagggcctgaatctgccagcccctcaggatctccttccctctgggccctccccagccttaaggagcctcccagacagaagggtggacagagccacctgggcagcccgagagacacacgggggtcctccctgtggacagccctgccagcttccgcccagccctgagcttcatttgcatcttgaggagtaaggggtggtgaaatgggaatgctggtctggctcagctggtcgtgggcataagtgcccgctgaatggatggcatctctccctcctgtcttatgttctggggtccaggtgcttcccagggccatgcccctgctgctaatgcttgccctaacccttaccctaaccagcgtccagcgtcgtctcaccgagccgtaaataaatcaacagattcgcattgtcaaaaaaaaaaaaaaaaaa

SEQ ID NO: 12 - DNA, 4028 bp, Mus musculus
ccacgcgtccgcccacgcgtccgcccacgcgtccgcccacgcgtccggcctacttggcctgcctgcgatgcggtaccaacaccgcacgaagtgtgtacagattcccagttagacagcaggagggacctgggagcggccagggggatgttttatctctaagagaccaagagctcaggcagggcttctgtgccctgcttcctccctggcttgagctggatcctggaccagctgctgacctcctgttcactctggcactgccctcacgtctccgtcatgacccatctgctcacagtgttcctggtggccctgatgggcctgcctgtggcccaggctctggagtgccacgtgtgtgcctacaatggagacaactgcttcaaacccatgcgctgcccagccatggccacctactgtatgaccacacgaacttacttcaccccataccggatgaaggtgaggaagtcctgtgtccccagctgctttgaaaccgtgtacgatggctattccaagcatgcatctgccacctcctgttgccagtactacctctgcaacggtgctggctttgctaccccggtgaccttggccctggtcccagcactcctagctaccttctggagcttgctgtaaagctcggttccccaagccagatccactcaaacgcaacactctcaaaaaacacagtttccctctctctcccaattcactccacccaacgctcttccttctgacactcctcaactaccacgaggtcccatggctacctacgaaagaactgatggcatccagatacctcactccaaggtcattttcagaaggctgacatgtggacctgtaatgtgcccacccatgggtggggcaggctgggcttctcctctacccaagatcaggggcatctgggagaatgtttatggaggaggggtcatcactcaagtcaaggagcactgatttgatagaattagtagccaaactccaccttcagaaccctgcctcagtctacccagtagaggatgggtctgctagaggtgaggggaggagagcggcggagaataacgagctggctagaagcagagaaagactcagcagggctgtctccgaagatcagcgcggcttgccagagcaaatgtgatgtggaagccatgtgaggaagccctttgtcatttccacttatctgaggaactctgccagacctgatgttgggatagccattggccaagggttcctagcaacggcgtcatttccataggccactgaaatccctccagccccagctcagcaggccccttgacctccactacagtccttcattcacacaccagctgctgggccttgaagttggcagggacttgggagcaggtgacccatgctattttttgtctggcctgttattctgggcatggcaagaagggatcagacgcaggtcagagcagggcagtagggcgactgagacagggaaacagacttcagccagtggcttcccaggtcccgtaggcagctcctacatccttcagtctcttgttacattcccgggagacaaatatacagggagccaagccgagtgctaggtgatgactgcctgtgaagtctattgtggccacagactgctgggtaccaagtctcaggagaacccagcctagatttaggagacacagatctgcctttcatgcagtgtagctgtccttgggagccttaccatgctctctaactagtacctcaactcacatgtcactgaggaaccccctaacactggcccagcccaggggtcgggatgctggccaatgtccatggagtgggactacccatggagagtccttgggtcatcacatcacaaatgttttattccaatctcccagtggtgagagctcgggacacaaaggtccatcctggggaccttcttcctggttctaggcagacctgaactctgtctgctgctagagctgatgtggttttccgcctcagtttcctcctccggggataggccaccggaggatttgggagggtggggagggcatcctgctgatgggctcgccgaggttctcaggaacaggaacgggcggggctttagtacacaggtgagttgggtgggaactggcccggagctgaggagacactgactgggcagagggaagatgagtctcaagggagggcaggaaaagggagggggagcgcgcatgcacatgtgcactcagtgcaggctacagagcccaaaaggcagcactggctgtggtgtcccctgaggcccaggcaagatgctaggaggaagccaatgctgcccccacctgagctcacatggaacatgcacaccaccagcagcagcagcaagcattgagactgacctgtggacgccatagggcactggcaaggagggtcagaggcgggtccctgactcagtgggtgaggcccgggaaacattatcctgttaccctgcgtgtgcaagatcattgtccccagctagatggcgtcctcaaccaaaactgagaggagccccagttcaggtcctccctcctaccacaagggggtggtgtggaggaggcttgattgcccttggagaagcaccggtactgcagagctgggggccagcttctttcatctgtgtctagacaccgaccagataggccccacagtggcaacactgccacacagccctacaagaagccctgtgcctagctagcacagagccccaaaaggtgctcaattaatacagggccaagcctgccagtgggggggatgcagattaggggaacagacccagatggcctgtcctgaaccctgtctggggtggtgtgatgagcatctgtctagcccactgcaggtggctctacacactccacaacagttctgcaaaagtgtatgaggtggtcattactgcgcccctctcacaggtaaaggcactgaggcacggaggagtgaggcacttcattttcctgggccattcaactttccaggaccaacacattcaactatgggtactactccaatagctggggttctttgaggctgggccccctgaagatgatagtggcttcatcaaccagagaatttcagagtgcagtgttgtaggagcctatgaacctgaaatgtcagaactggaggtttgaggggctgaggggtaggccaggggtgtctggccccttgtgtggagacagagagagagggaacatgggatggggtagtagagagaagtgcaaaggagcgtcagcctttctcagggctaatgctgtcagggacgagggctcaagcctgtgagtgttctcacactgtgataaacagtggcccctcaacacagacggtgtccagagtggccggcagtggttatctagagttgcaatctggaagcctcttggtagtcactggagagaggccgcttgatgggacagcaccaaatgtgtgtgcttctgtgggatgtgaggaagctgggtcagcgcatgaagccaaagcgtccttcagagcagaggggtggctggtctagtccaccagagacaagctatccagtgagagtcatactctgccaccgtctctgtgattaccttaccccaaagcagacggggacgggatgcagagcacccgtgtcttcatcttctgcggcaagcacgtgagttcacattctgaaactctagaaagatttccaggagtggggtgtgcctttgctttggtgcatggttacttcctggcaagcaccgtggcatcccgcagcactgagtgacctgggctcctcaagccatctcattggtgaaatgacagtgccagtaccctctcagctggctcttggaggcctgtgcatggggtctgcacagaggaggcccccaaactatgcatggacggacacgtgatgcctagcacttcccttggttgtgtctctgccaaccccaggctctcacccagcaaggaaatgaaatccacttttatgacacatctccctcccccagccagctccattcacctatatgccagggtggtccctttcaatgtctgtcccccattggatgaataaacaagcgaaggacaaaaaaaaaaaaaaaaaaaaaaaaaaaaaa

SEQ ID NO: 13 - DNA, 1679 bp, Homo sapiens
tcagccgccaactccggaggcgcggtgctcggcccgggagcgcgagcgggaggagcagagacccgcagccgggagcccgagcgcgggcgatgcaggctccgcgagcggcacctgcggctcctctaagctacgaccgtcgtctccgcggcagcagcgcgggccccagcagcctcggcagccacagccgctgcagccggggcagcctccgctgctgtcgcctcctctgatgcgcttgccctctcccggccccgggactccgggagaatgtgggtcctaggcatcgcggcaactttttgcggattgttcttgcttccaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcaggccagggctccgcaccaccatcctgttcctcaaattagccctcttctcggcacactgctgaagctgaaggagatgccaccccctcctgcattgttcttccagccctcgcccccaaccccccacctccctgagtgagtttcttctgggtgtccttttattctgggtagggagcgggagtccgtgttctcttttgttcctgtgcaaataatgaaagagctcggtaaagcattctgaataaattcagcctgactgaattttcagtatgtacttgaaggaaggaggtggagtgaaagttcacccccatgtctgtgtaaccggagtcaaggccaggctggcagagtcagtccttagaagtcactgaggtgggcatctgccttttgtaaagcctccagtgtccattccatccctgatgggggcatagtttgagactgcagagtgagagtgacgttttcttagggctggagggccagttcccactcaaggctccctcgcttgacattcaaacttcatgctcctgaaaaccattctctgcagcagaattggctggtttcgcgcctgagttgggctctagtgactcgagactcaatgactgggacttagactggggctcggcctcgctctgaaaagtgcttaagaaaatcttctcagttctccttgcagaggactggcgccgggacgcgaagagcaacgggcgctgcacaaagcgggcgctgtcggtggtggagtgcgcatgtacgcgcaggcgcttctcgtggttggcgtgctgcagcgacaggcggcagcacagcacctgcacgaacacccgccgaaactgctgcgaggacaccgtgtacaggagcgggttgatgaccgagctgaggtagaaaaacgtctccgagaaggggaggaggatcatgtacgcccggaagtaggacctcgtccagtcgtgcttgggtttggccgcagccatgatcctccgaatctggttgggcatccagcatacggccaatgtcacaacaatcagccctgggcagacacgagcaggagggagagacagagaaaagaaaaaca

SEQ ID NO: 14 - DNA, 937 bp, Homo sapiens
ccggaggcgcggtgctcggcccgggagcgcgagcgggaggagcagagacccgcagccgggagcccgagcgcgggcgatgcaggctccgcgagcggcacctgcggctcctctaagctacgaccgtcgtctccgcggcagcagcgcgggccccagcagcctcggcagccacagccgctgcagccggggcagcctccgctgctgtcgcctcctctgatgcgcttgccctctcccggccccgggactccgggagaatgtgggtcctaggcatcgcggcaactttttgcggattgttcttgcttccaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcaggccagggctccgcaccaccatcctgttcctcaaattagccctcttctcggcacactgctgaagctgaaggagatgccaccccctcctgcattgttcttccagccctcgcccccaaccccccacctccctgagtgagtttcttctgggtgtccttttattctgggtagggagcgggagtccgtgttctcttttgttcctgtgcaaataatgaaagagctcggtaaagcattctgaataaattcagcctgactgaattttcagtatgtacttgaaggaaggaggtggagtgaaagttcacccccaaaaaaaaaaaaaaaaaaa

SEQ ID NO: 15 - DNA, 1890 bp, Homo sapiens
ccttcagtctcagccgccaactccggaggcgcggtgctcggcccgggagcgcgagcgggaggagcagagacccgcagccgggagcccgagcgcgggcgatgcaggctccgcgagcggcacctgcggctcctctaagctacgaccgtcgtctccgcggcagcagcgcgggccccagcagcctcggcagccacagccgctgcagccggggcagcctccgctgctgtcgcctcctctgatgcgcttgccctctcccggccccgggactccgggagaatgtgggtcctaggcatcgcggcaactttttgcggattgttcttgcttccaggctttgcgctgcaaatccagtgctaccagtgtgaagaattccagctgaacaacgactgctcctcccccgagttcattgtgaattgcacggtgaacgttcaagacatgtgtcagaaagaagtgatggagcaaagtgccgggatcatgtaccgcaagtcctgtgcatcatcagcggcctgtctcatcgcctctgccgggtaccagtccttctgctccccagggaaactgaactcagtttgcatcagctgctgcaacacccctctttgtaacgggccaaggcccaagaaaaggggaagttctgcctcggccctcaggccagggctccgcaccaccatcctgttcctcaaattagcctcttctcggcacactgctgaactgaaggagatgccaccccctcctgcattgttcttccagccctcgcccccaaccccccacctccctgagtgagtttcttctgggtgtccttttattctgggtagggagcgggagtccgtgttctcttttgttcctgtgcaaataatgaaagagctcggtaaagcattctgaataaattcagcttgactgaattttcagtatgtacttgaaggaaggaggtggagtgaaagttcacccccatgtctgtgtaaccggagtcaaggccaggctggcagagtcagtccttagaagtcactgaggtgggcatctgccttttgtaaagcctccagtgtccattccatccctgatgggggcatagtttgagactgcagagtgagagtgacgttttcttagggctggagggccagttcccactcaaggctccctcgcttgacattcaaacttcatgctcctgaaaaccattctctgcagcagaattggctggtttcgcgcctgagttgggctctagtgactcgagactcaatgactgggacttagactggggctcggcctcgctctgaaaagtgcttaagaaaatcttctcagttctcccttgcagaggactggcgccgggacgcgaagagcaacgggcgctgcacaaagcgggcgctgtcggtggtggagtgcgcatgtacgcgcaggcgcttctcgtggttggcgtgctgcagcgacaggcggcagcacagcacctgcacgaacacccgccgaaactgctgcgaggacaccgtgtacaggagcgggttgatgaccgagctgaggtagaaaaacgtctccgagaaggggaggaggatcatgtacgcccggaagtaggacctcgtccagtcgtgcttgggtttggccgcagccatgatcctccgaatctggttgggcatccagcatacggccaatgtcacaacaatcagccctgggcagacacgagcaggagggagagacagagaaaagaaaaacacagcatgagaacacagtaaataaataaaaccataaaatatttagcccctctgttctgtgcttactggccaggaaatggtaccaatttttcagtgttggacttgacagcttcttttgccacaagcaagagagaatttaacactgtttcaaacccgggggagttggctgtgttaaagaaagaccattaaatgctttagacagtgt

SEQ ID NO: 16 - DNA, 1797 bp, Mus musculus
agaagaggcgagacttttttgggtgctccggatcgccagtagttcttcaagcctcagcagccaactcctccggaggcgctgcgctccgccccagggagcgcgaatccaaggagcctgggaccagcctctgggagcccccggcgcgggcgatgcgggcgccgcgggcgacacctgcgactcctctcggtggcagccgtcgcttgggcggcagcagcgcgagcctcggcagcctcggcagctactgtcgccgcggccagaacagcctccgctgcggtcgtggtctctgatgctcttgcccgctcccggccctgccgatccgggaggatgtgggttctcggcatcgcagcaactttttgcggattgttctggcttccagggctggcgctgcaaattcagtgctaccagtgtgaagaattccagctgaacaacgattgctcatcccctgagttcatcgtaaattgcaccgtgaacgttcaagacatgtgtcagaaagaagtgacggagcaaagtgctgggatcatgtaccggaagtcgtgtgcatcgtcagcagcctgtctcattgcttcagctgggtaccagtccttctgttcccctgggaaactgaactccgtgtgcatcagctgctgcaacacccctctttgcaatgggccgaggcccaagaagagaggcagctctgcctcggccatcaggccagggcttctcaccactctcctgttcttccacttagccctctgcttggcacactgctgaagctaaaggagatgccaacccctgctgcctcacctgtctggtccttcgtctctcaccttcccgagtctcttctgggtgtccttttattctgggtagacaagggagtctttttgttccctcctttcaagtaacgcaagattgccgtgcacaaatacttttgtaagctctgaaccaattcattctgaatttctgtgtgtagttgaagaaaaaagcatggagcagaaagtccagaccctcccatcccaatctggttaaccaccgccaaggctagcctggaagaaccagcccttagaagtcattgagatacgcatctgcctttcccaaagccttgagcttccattctgtcccagtaggagtcacagtctattcagagactgctgctgcgtgaaggtaactttgcttttgcgggaggggagagccagtttcggctcaaggcttctgaacttgccattcatacttcctgctcctgtaaactattttctggggtggacccagctggtttggtctctgagccagtctgtggtgactcaggactcaagggctggggcttagcctctccaggcttggcctcagtctgaaaagtgcttaagaaaaccttgttagttctcctggaggaagagttactgcgccgggaggctaggaagatgagggggctgcgggctgagctggtgctgtccttggtggagatgaagcgggcacgctggcgtttctcttggttggcatgctgcagagtcaggcggcagcagagcacctgccagaacaccttccggaactgctgagaggactcgttgtagaggagagggttgaccacagagctgaggtagaagaaggtatcagagaagggcaggaggatcatgtatgccctgaagtacgttctggtccagtcatgtttgggttttgctgcagccatgatccgtcggatctgattgggcatccaacacacggccaacgtcaccacaatcagtcctggcaggcaagaacaggagagaaaaggagacggggagagaaacagcatgagaacaaaaaaaaaaaaaaaaaaaggccaca

SEQ ID NO: 17 - DNA, 550 bp, Homo sapiens
ccagtctgtcgccacctcacttggtgtctgctgtccccgccaggcaagcctggggtgagagcacagaggagtgggccgggaccatgcgggggacgcggctggcgctcctggcgctggtgctggctgcctgcggagagctggcgccggccctgcgctgctacgtctgtccggagcccacaggagtgtcggactgtgtcaccatcgccacctgcaccaccaacgaaaccatgtgcaagaccacactctactcccgggagatagtgtaccccttccagggggactccacggtgaccaagtcctgtgccagcaagtgtaagccctcggatgtggatggcatcggccagaccctgcccgtgtcctgctgcaatactgagctgtgcaatgtagacggggcgcccgctctgaacagcctccactgcggggccctcacgctcctcccactcttgagcctccgactgtagagtccccgcccacccccatggccctatgcggcccagccccgaatgccttgaagaagtgccccctgcaccaggaaaaaaaaaaaaaaaaa

SEQ ID NO: 18 - DNA, 507 bp, Mus musculus
agacataacccaaacagaataacctgaaagagacccacgaccatgcaggggacctggatggtgctgttggcactgatattgggcaccttcggggagcttgctatggccttacagtgctacacctgtgcgaatcctgtgagtgcatccaactgtgtcaccaccacccactgccacatcaatgaaaccatgtgcaagactacgctctactccctggagattgttttccctttcctgggggactccacggtgaccaagtcctgcgccagcaagtgtgagccttcggatgtggatggcattgggcaaacccggccagtgtcctgctgcaattctgacctatgcaacgtggatggggcacccagcctgggcagtcctggtggcctgctccttgccctggcacttttcttgctcttgggtgtcctgctgtaaagccatggccatctagctccactcccttgtccctgacatcccagttccctaatgcctagaagaaatacaatggccatctgc

SEQ ID NO: 19 - DNA, 4720 bp, Homo sapiens
ggggccggggcaggcctggggcggggcctgcggtgagctcgcgctgggcgggctgttccgggggcgggactgggctgggcctgcggtgagcatcaggcgatgcggcacgggtgctgcgggacacacagacacgcctacgattagactcaggcaggcacctaccggcgagcggccgcgggtgactcccaggcgcggcggtacctcacggtggtgaaggtcacagggttgcagcactcccagtagaccaggagctccgggaggcagggccggccccacgtcctctgcgcaccaccctgagttggatcctctgtgcgccacctgagttggatccagggctagctgctgttgacctccccactcccacgctgccctcctgcctgcagccatgacgcccctgctcaccctgatcctggtggtcctcatgggcttacctctggcccaggccttggactgccacgtgtgtgcctacaacggagacaactgcttcaaccccatgcgctgcccggctatggttgcctactgcatgaccacgcgcacctactacacccccaccaggatgaaggtcagtaagtcctgcgtgccccgctgcttcgagactgtgtatgatggctactccaagcacgcgtccaccacctcctgctgccagtacgacctctgcaacggcaccggccttgccaccccggccaccctggccctggcccccatcctcctggccaccctctggggtctcctctaaagcccccgaggcagacccactcaagaacaaagctctcgagacacactgctacaccctcgcacccagctcaccctgcctcaccctccacactccctgcgacctcctcagccatgcccagggtcaggactgtgggcaagaagacacccgacctcccccaaccaccacacgacctcacttcgaggccttgacctttcgatgctgtgtgggatcccaaaagtgtccggctttgatgggctgatcagcccctcgcctgtccagggctatttatggggaggggcccagtcaggcagggagcactgagcagatgggagctgtggccaagcccaccttctgggaagccctaggagaggcccctgcctcagtctgcccctggtgtgctggggcagggggaagacaaggaatgcccgcaggtggggtggtggggagactcttaggaggaaaggctcctccaggcctagtcacgcttcctactgaggccaggagctgccaggaccggtacgggatcagggctgtgggaggagggcctttggcaccggcccctgtgctaggaagtctgccaggcccgagttggagccaccccctgcaggggagggggcggctctgcctcagcaggccccagggcccccgaagtcacagaagctttttcgggtccagcaaggggtgtgtgtcctctcagtcaaaccccttgacgtttcccaccccctcacggggagggcaccaggcctgaagctggcaggagctagggccatgctatatttggtgggtcctggacgctgacccggccagcgctattctgggcagggagggaaaggggcagagcaggtggtcccccgagtcctggtccccaaccacagcaggacccagccgagcaaggcaaaagacgcaggactgggggatgcgcgcacaggctgggggttgggagcagcctggggccggcgcgggcctgggcgtgggaaggcggagcatgccaccctctcgtggccgtgcgggggcgggagtggggagggtggggtggggagggcggagacccagcccctcccccgcagcgggatgcgcacagtgagtgggtccctccatcaccttccacctggggatgcacccactgggagggcagggtggaagccccagctgggtgtgtggactcccagggaccccaccccaggcctgggaagcaggggtcagcccaacacgcaccaatccatccccgatgcaggtagcccagggagcacctgcccctgctgtgaatggggcattcgggggcgtgagaccttttggtgccagcggtcacgctgcacctgaggcccccacctgaccagtgctcccagctctggtgtcctgagaaacccttcaagccatcccgcatgggcaggatggtgacatatccatgtggtaggattgtcccggccccaaagtatggccctggtcaggggagcccctgctggaaattgcatctccagagctttgatgcaggacccctgggggatcagggaatgagggtctccaccccaggggtctccttgcagtgagtctatatgcaggcctgcgttctgctcctggggctggttctgagtgcccagcttcagtctcctgagaacatgaggatgggagggggcagagtcttgctgagggcacacccagttcccgctggaggaggacagtgccagtcttctgcaaagggaccttgggtgggaacgggcccggagcgggaggaacgtgactccccagagggaagatgggcatcatactgggcccagagctgggaaggagttgctgccagcacagggtgggcctggactcccctcgcccctacccccagtggttgtggctgtagccctaagcctggagagcaggaccggcccggggtgtctgggaggctgccaggtgcctcccagagctcccaagggcccccacctgcaagtgccagcctcagggcagtgcccaaatgaggccctctcagctgcagccagcgatgccttgggatgcttaccgggagggaggcggctttgggctcctaagtccttgggagaggctgggagcagtcactgcgcggcttgcgcaagcccattgtcgggttgggtggcttcctcagccagggctgggagggactccaggatcaggtcctccctgtctcgagtctcagtggggtgatggggaggagacctggccacccatggctcaggggcagctgagaacaaggacctgctggagctggaagtgctgtggtgttgaggggtggggtgggcagcttctcacacctgcctcctgcctccttctgtccacctttccaccaccctgacctgtcccagccccacacatggttctgcctggctggcctgcccttggcacctggcgtagagcacacagaaggcactcagctaatgctgggcaggcccactcatggggagtgcgtggctgtgcagcaccagggaaccggcacagcagcgccggcagaaatcacagcagtaaacttgtccgggttgtatgcatcaaggtggcgatggacgtgggtccccccactgcactgtggccctgagcactgtatagcagcccggcaatgggagccattatcttgcccctttgacagaggaggacacagaggcacagggaggtgaagtagctgccccacactagtgcctcctcgctcactcaccaccccctgcaccacagtgcagccgcttctcccaccagctggggttccttggacccccaagcctgggaagggggaggtgagtttacaaaatggaaagcttaaaaggagaaaagtggaaccagaggtttgagaagccctgagtggtagagtaaggcctccagcgctgcctctgggtgcagggcagagtggcagaggagagggggagaggcactgggcaccatgggggcccagttcccacttcggggatctctctcgcagaaccgagggtccccttcatgggggtagatgcccagggctagctgttgccactgtctgtgtggacctgagtcctggacatgcccgagtgactcaggagtggctgcttgggcgggctctgtcaccctaggatgttatacattctgggaactggacaggagtggctgcttgggcgggctctggcaccctgggatgttatacattctgggaactggacaggagtggctgcttgggtgggctctggcaccctgggatgttatacattctgggaactgcaatcagccactagagaagtcggagctacaggaagtgaccctggggtgggacctggggacatggccaggtcagcatggggacacccggctccagcaggagctctggtctgtcctggggtctttgggggcagggctgcggccctgggcaggcttcctccaggcggaggtcctggggaagtgggggagccaggccagctgccgcctcccccactatgtagcatctgattcgtcatctctcatgaaggcgatttggttcataactctgaaactctgaaaaaggtcaaaagaagcagagaggccctcggtggatatgccagcttttctgccggtgctttctcccactactctgggtggtctgctctcctcttcaaacctcagctcgcagggagggcctgaatctgccagcccctcaggatctccttccctctgggccctccccagccttaaggagcctcccagacagaagggtggacagagccacctgggcagcccgagagacacacgggggtcctccctgtggacagccctgccagcttccgcccagccctgagcttcatttgcatcttgaggagtaaggggtggtgaaatgggaatgctggtctggctcagctggtcgtgggcataagtgcccgctgaatggatggcatctctccctcctgtcttatgttctggggtccaggtgcttcccagggccatgcccctgctgctaatgcttgccctaacccttaccctaaccagcgtccagcgtcgtctcaccgagccgtaaataaatcaacagattcgcattgtcaaaaaaaaaaaaaa